(19) United States
(12) Patent Application Publication
(10) Pub. No.: US 2011/0253139 A1
(43) Pub. Date: Oct. 20, 2011

(54) INHALER MODULE AND RELATED SYSTEM

(75) Inventors: Warren E. Guthrie, West Olive, MI (US); David L. Klamer, Zeeland, MI (US); Kent D. Pilcher, Holland, MI (US); Gordon J. Stannis, Saugatuck, MI (US)

(73) Assignee: SPECTRUM HEALTH INNOVATIONS, LLC, Grand Rapids, MI (US)

(21) Appl. No.: 13/087,415

(22) Filed: Apr. 15, 2011

Related U.S. Application Data
(60) Provisional application No. 61/324,613, filed on Apr. 15, 2010.
(60) Provisional application No. 61/324,617, filed on Apr. 15, 2010.

(51) Int. Cl. A61M 15/00

(52) U.S. Cl. 128/203.14; 128/203.12

(57) ABSTRACT

Apparatuses and methods for mounting an inhaler module to an inhaler, indicating compliance with a treatment regiment, shaking an inhaler and indicating mixing status, and reminding a user to prime the inhaler. The mounting element can have an elastic body adapted to interchangeably join both an inhaler and an inhaler module. Compliance can be indicated at a glance for a caregiver by providing an indication of the level of asthma medication treatment compliance. An accelerometer can be used to track mixing status of the medication canister and timestamped accelerometer data can be used to provide appropriate reminders to prime the inhaler.

### FIELD OF THE INVENTION

The present invention relates generally to inhalers, and more particularly to a device that monitors compliance with an asthma medication regimen.

### BACKGROUND OF THE INVENTION

An inhaler is a device that provides controlled oral or intra-nasal administration of medication to a patient, and is typically used to treat asthma, allergies and other respiratory ailments or diseases. Many physicians direct patients to use an inhaler on a regular basis, under a prescribed compliance regimen.

Although an inhaler can be an effective tool in treating asthma or allergy symptoms, the inhaler is only effective if properly maintained and used consistently. Patient compliance with inhalers is traditionally low, particularly in younger patients because of forgetfulness, a lack of social support, parent and child concerns about medication safety, and social stigma. Additionally, patient compliance with both maintenance and prescribed compliance regimens is difficult for a physician to monitor.

When an inhaler is new or has not been used for a while, the medication may separate from the other ingredients in the canister and the metering chamber. Shaking the inhaler will mix the ingredients in the drug reservoir but may not produce enough turbulence to reblend the ingredients in the metering chamber. Priming, or releasing one or more sprays into the air, ensures the next doses will contain an accurate amount of medication.

A physician may rely on the patient's recollection of whether the inhaler was properly maintained and used at the appropriate, prescribed intervals. Because data obtained from the patients may be inaccurate, it can be difficult for the physician to draw any meaningful conclusions with respect to the effectiveness of the medication and dosage amount for an individual patient. It may be even more difficult to identify trends based on data from a group of patients.

To address issues with patient compliance in a medication regimen, several asthma compliance monitoring devices have been developed. Two examples are the device in U.S. Pat. No. 6,202,642 to McKinnon and the inhaler in U.S. Pat. No. 5,363,842 to Mishelevich.

McKinnon includes an electronics module that is connected to a metered dose inhaler (MDI) via a highly specialized sleeve adapter and heel adapter that wrap around different portions of an MDI. By way of the physical interaction of the sleeve adapter and heel adapter, the electronics module detects when the inhaler is activated to administer a dose, and thereby monitors different factors related to compliance, including dose delivery time and the number of doses taken during a time frame. A physician can review the data collected by the device to evaluate compliance with a prescribed compliance regimen.

While the sleeve adapter and heel adapter adequately join the inhaler module to the MDI, the construction suffers several shortcomings. First, the sleeve adapter and heel adapter must be specifically configured to connect to the contours of the MDI. Because there are many MDIs on the market, with more being added every year, a wide variety of differently configured sleeve adapters and heel adapters must be made to accommodate the different inhalers. In many cases, it simply is not commercially feasible to make such a variety of adapters. Therefore, many patients may not have access to such a compliance monitoring inhaler. Second, the sleeve adapter is configured so that it includes the power source for the electronics module. This can be an issue where a patient desires to remove the electronics module from the MDI and review information stored on the device. Third, the sleeve adapter and heel adapter tend to be quite bulky. For younger patients, such as children, this can present issues with the way they grasp and actuate the inhaler.

In addition, many asthma compliance monitoring devices are configured to mount to a docking station, where they download the data captured while in use on an MDI. Most docking stations include data transfer terminals that facilitate the download of data to the docking station, and subsequently to a network, and eventually to a physician's computer. While such conventional docking stations provide adequate data transfers, they fail to provide any indication as to whether the patient is complying with the compliance program. Thus, a parent or caregiver of a patient can go for weeks or months without knowing whether the patient has been adhering to their compliance regimen—until they are informed by a physician that no data has been transferred to the physician's computer from the patient's device. Accordingly, a caregiver might not have the information to help the patient better follow the compliance regimen. This can potentially put the patient at a higher risk of complications or a serious asthmatic attack.

Mishelevich discloses a highly specialized metered dose inhaler (MDI). The inhaler includes a motion sensor, a microprocessor, a pushbutton switch, indictor lights, an actuation sensing switch, a pressure-sensor diaphragm, and other circuitry. The motion sensor can detect when the inhaler has been shaken so that power can be provided to the circuitry. The actuation sensing switch detects when the inhaler is activated to administer a dose and sends a signal to the microprocessor. The pressure-sensor diaphragm senses air flowing through the inhaler and sends a signal to the microprocessor. As with McKinnon, the data is used to monitor different factors related to compliance so that a physician can review the data collected by the device to evaluate compliance with a prescribed compliance regimen.

While the various buttons and sensors provide adequate user interface and compliance data collection, the construction suffers several shortcomings. First, the inhaler must be specifically configured to accept specific medication canisters. Because there are many different shapes and sizes of medication canisters on the market, with more being added every year, a wide variety of differently configured inhalers must be made to accommodate the different inhalers. In many cases, it simply is not commercially feasible to make such a variety of different inhalers with these circuit components. Further, it may be cost prohibitive or inconvenient for users to purchase and track multiple inhalers for different shapes/sizes of medication. Second, because of the physical interaction between the actuation sensor and the canister, the inhaler is more prone to malfunction over time. This can lead to users having to replace the entire inhaler, despite the actuation sensor being the only malfunctioning part. Third, the additional components integrated into the inhaler tend to make the inhaler bulkier.

### SUMMARY OF THE INVENTION

There are at least four main aspects for monitoring compliance with a medication treatment regimen. First, various embodiments are provided for mounting an inhaler module to an inhaler. Second, various embodiments are provided for indicating, at a glance, levels of compliance with a medication treatment regimen to a user or caregiver. Third, various embodiments are provided for tracking and indicating the mixing status of the contents of the medication canister based on accelerometer output. Fourth, various embodiments are provided for reminding a user to prime the inhaler at the appropriate times.

In the first aspect, a mounting element for an inhaler module can be provided including an elastic body that automatically conforms to the contours of one or more inhalers to physically join the inhaler module in a sturdy and consistent manner with the inhaler. A docking station can be provided including an indicator that indicates to a care giver that: (a) a patient has used their inhaler at a compliance level relative to a predetermined treatment regimen; (b) compliance information has been or is being downloaded to the docking station; and/or (c) the power source for the inhaler module is charging or charged, among other things.

In one embodiment of the first aspect, the mounting element is constructed from an elastomeric material, such as rubber, ethylene-vinyl acetate (EVA), thermoplastic elastomers (TPE), thermoplastic polyurethane (TPU), or other elastomeric materials. Optionally, the mounting element is an elastomeric material that is durable, latex free and capable of stretching up to two, three and even four times its original dimensions to fit over a variety of conventional inhalers to secure the inhaler module to those inhalers.

In another embodiment of the first aspect, the mounting element can include a band that is adapted to align with and surround a vertical portion of a conventional metered dose inhaler. An attachment element, with an attachment projection defining a recess between an attachment element base and a flange member, can be joined with the inhaler module. The flange member can be adapted to fit through a hole in the mounting element when the periphery of the hole is stretched to join the mounting element with the attachment element in order to join the inhaler module and the mounting element.

In another embodiment of the first aspect, a method for installing an inhaler module on an inhaler with the mounting element is provided. The method can include the following steps: providing an inhaler; providing an inhaler module; providing a mounting element including an elastic body with an internal cavity and a hole; providing an attachment element joined to the inhaler module, where the attachment element includes an attachment projection that defines a recess between an attachment element base and a flange member; stretching and inserting the flange member into the mounting element hole; allowing the periphery of the hole and the elastic body to return to at least a partially unstretched state so that it grips or generally surrounds the attachment element base; stretching a portion of the elastic body to increase the dimensions of an internal cavity of the mounting element; inserting a portion of an inhaler into the increased dimension of the internal cavity; allowing the elastic body to relax somewhat from a stretched state so that it exerts a gripping force on at least a portion of the exterior of the inhaler; and optionally moving the elastic body to position the inhaler module in a predetermined spatial location relative to the inhaler.

In another embodiment of the first aspect, the mounting element can include a mounting base that joins the inhaler module with the elastic body. The mounting base can include one or more attachment elements that attach to surface contours of, or corresponding fasteners on, the inhaler module. For example, the inhaler module can define a peripheral groove. The mounting base can include one or more elastic bands that interfit in the groove. When interfitted in the groove, the bands can lock the inhaler module in a predetermined spatial configuration relative to the inhaler to which the mounting element is joined.

In another embodiment of the first aspect, the mounting element can include a first band or first portion that is adapted to align with and surround a vertical portion of a conventional metered dose inhaler, and a second band or second portion that is adapted to align with a mouth piece of the inhaler. The first and second bands or portions can be transverse to one another and can define a contiguous internal chamber that generally conforms to many of the exterior surfaces of the inhaler.

In yet another embodiment of the first aspect, the mounting element can be constructed from the elastomeric material and of a dimension sufficient to exert a gripping force on the first and/or second portions of the inhaler so that an inhaler module attached to the mounting element is held in a predetermined spatial relationship relative to the actuator. The mounting element can be constructed such that in normal activity and storage, the inhaler module is prevented from moving, or only moves minimally relative to the inhaler.

In yet another embodiment of the first aspect, the mounting element can include a first band that circumferentiates the first portion of the inhaler and a second band that circumferentiates the second portion of the inhaler. The first and second bands can be integral with one another or they can be separate and distinct from one another.

In still yet another embodiment of the first aspect, the elastic body can define an internal cavity aligned with a longitudinal axis of the elastic body, within which a longitudinal or vertical portion of an inhaler is adapted to fit. The elastic body can also include an opening that opens generally orthogonally to the internal cavity. A mouthpiece of an inhaler can fit through the opening and can be accessible for use.

In yet a further embodiment of the first aspect, the elastic body can be joined with a mounting base including a peripheral portion that is adapted to engage an outer surface or periphery of the inhaler module. The inhaler module can be constrained within the peripheral portion so that the periphery of the inhaler module is restrained relative to the mounting element, and held in a predetermined spatial configuration relative to the inhaler to which the mounting element is joined.

In still yet a further embodiment of the first aspect, the mounting base can include one or more fasteners that join with corresponding fasteners on the inhaler module to join the inhaler module with the mounting element.

In another embodiment of the first aspect, a method for installing an inhaler module on an inhaler with the mounting element is provided. The method can include the following steps: providing an inhaler; providing a mounting element including an elastic body; stretching a portion of the elastic body over a portion of an inhaler module adapted to record dose information collected relative to an inhaler; stretching a portion of the elastic body to increase the dimensions of an internal cavity of the mounting element; inserting a portion of an inhaler into the increased dimension of the internal cavity; allowing the elastic body to relax somewhat from a stretched state so that it exerts a gripping force on at least a portion of the exterior of the inhaler; and optionally moving the elastic body to position the inhaler module in a predetermined spatial location relative to the inhaler.

Further optionally, after the medication in one inhaler is depleted, the elastic body and inhaler module can be removed from that inhaler. Even further optionally, the elastic body can be stretched again to fit on another inhaler using the same process identified above in connection with the previous inhaler. In such a manner, the mounting element, and subsequently the inhaler module, can be used on multiple inhalers.

In one embodiment of the second aspect, a docking station can be provided and an inhaler module can be positionable in the docking station to download compliance data from the inhaler module to the docking station. The docking station can include a microcontroller that selectively communicates with the inhaler module to transfer data to and from the inhaler module. The docking station can include one or more indicators. The indicators can be in the form of one or more visual or audible indicators that indicate levels of medication treatment compliance using the inhaler. The indicator can provide a caregiver a visual or audible output that is readily discernable to determine the level of medication treatment compliance by the user of the inhaler.

In another embodiment of the second aspect, a method is provided for monitoring compliance and outputting signals concerning compliance levels, status of the inhaler, and/or the charging of the inhaler module. The method can include: providing an inhaler module that mounts piggyback to an inhaler with a mounting element; storing asthma medication treatment compliance data related to the dispensing of medication from the inhaler with the inhaler module; downloading the asthma medication treatment compliance data from the inhaler module to a docking station; comparing the asthma medication treatment compliance data to pre-selected values for the asthma medication treatment compliance data; outputting a first signal via an indicator joined with the dock that is indicative of the level of asthma medication treatment compliance by a user of the inhaler.

In another embodiment of the second aspect, the docking station indicators can indicate the amount of medication left in the inhaler, whether the inhaler is past an expiration date, and/or the charging status of a battery of the inhaler module. The indicator can provide a caregiver a visual or audible output that is readily discernable to determine the status of any of the foregoing. The audible alert can also be used to let the patient know it is time to use their prescribed daily inhaler.

In another embodiment of the second aspect, a method is provided for monitoring compliance and outputting signals concerning compliance levels, status of the inhaler, and/or the charging of the inhaler module. The method can include: providing an inhaler module; providing a docking station including a microcontroller and an indicator; docking the inhaler module in the docking station; providing a first signal if the inhaler to which the inhaler module was or is connected has been used at a predetermined compliance level relative to a treatment regimen; providing a second signal if the inhaler to which the inhaler module was or is connected is low on medication or the inhaler is past an expiration date; and providing a third signal when the inhaler module is downloading compliance data to the docking station.

In one embodiment of the third aspect, an apparatus is provided with an inhaler module, a mounting element, an indicator, and a microprocessor. The inhaler module collects information related to use of the inhaler. When the inhaler module is mounted on the inhaler, shaking the apparatus simultaneously mixes the contents of the medication canister and shakes the accelerometer. The microprocessor is programmed to 1) determine a mixing status of the contents of the medication canister based on the accelerometer output signals and 2) control the indicator to indicate the mixing status to the user of the apparatus.

In another embodiment of the third aspect, the inhaler module does not have any electrical or mechanical communication with the inhaler. The inhaler module does not interfere with the usage of the inhaler, but still provides instructions to the patient and collects information about use of the inhaler.

In another embodiment of the third aspect, the accelerometer output signals can be used to wake the inhaler module out of a sleep state.

In yet another embodiment of the third aspect, the indicator is a display and the mixing status of the medication can be indicated to a user via an animation on the display. The speed of the animation can vary as a function of the shaking such that the animation completes at approximately the same time in which the mixing of the medication is completed.

In the fourth aspect, an inhaler with a priming reminder feature is provided where the inhaler module can track when the inhaler is used, determine whether the inhaler needs to be primed, and can indicate to a user that the inhaler needs to be primed.

In one embodiment, the inhaler module is provided with an accelerometer, a controller, a memory, and an output. The inhaler module can record timestamps associated with shaking the inhaler. The inhaler module can use the timestamps to determine when the inhaler was last used and determine whether the inhaler should be primed, for example because the inhaler has gone unused for too long for a particular medication. This can help prevent both under-priming and over-priming of the inhaler.

In one embodiment of the fourth aspect, a method for operating an apparatus for monitoring compliance with a medication treatment regimen with a priming reminder is provided. The method can include the following steps: pairing an inhaler module with the inhaler, including configuring the inhaler module as a function of the inhaler medication; shaking the inhaler to mix the inhaler medication in the medication canister before each use; recording a timestamp in response to the shaking before each use; determining whether the inhaler needs to be primed based on the recorded timestamps and the inhaler medication; and in response to determining that the inhaler needs to primed, prompting the user to prime the inhaler.

In another embodiment of the fourth aspect, a method for operating an inhaler is provided. The method can include the following steps: providing an inhaler with a particular medication; pairing an inhaler module with the inhaler, including configuring the inhaler module as a function of the inhaler medication; shaking the inhaler to mix the medication; outputting a health related inquiry; accepting an answer to the health related inquiry; recording a timestamp associated with at least one of shaking the inhaler and accepting the answer to the health related inquiry; determining whether the inhaler needs to be primed based on the recorded timestamp and the inhaler medication; and in response to determining that the inhaler needs to primed, prompting the user to prime the inhaler. In one embodiment, determining whether the inhaler needs to be primed is based on the time differential between the last use, which can be a recorded timestamp, and the internal clock. For example, if n number of days have passed since the last use, the inhaler module can prompt the user to prime the inhaler. ‘n’ can be determined by user at set up or can be pre-programmed in software. The inhaler can also indicate that the inhaler does not need to be primed in response to a determination that the inhaler does not need to be primed. In some embodiments, if the inhaler does not need to be primed, the user will not be prompted.

In one embodiment, the inhaler module is utilized in conjunction with a docking station. The inhaler module can interface with the docking station to recharge the battery and transfer information. The inhaler module can transfer collected data to the docking station which can transfer data to a database located on a network, where the user or a third party reviews and optionally can transfer data back to the device. The dock can facilitate data transfer between the inhaler module and the internet through at least: a USB connection to a computer, Wi-Fi, cellular modem, or a standard phone line modem. The collected data may be aggregated and organized into a compliance report. The compliance report may include any of the inhaler module input events, such as the shake sensor output, input to health related inquiries or confirmation questions. The compliance report may include additional time stamped external data in conjunction with the timestamped inhaler module data to illustrate trends. The compliance report can include time differential data that indicates the difference between various timestamps recorded on the inhaler module. Other data can be mashed up with the inhaler module data to provide the compliance report. For example, the compliance information can be overlaid with other data, such as weather, pollen counts, or air quality data to help provide a more complete picture of the patient's health. The compliance report can be hosted on a server and made available over the Internet or another network. The compliance report or the underlying data can be made available to the inhaler user or health care professionals in a simple and intuitive manner, after logging into a secure web site or by print out.

In another embodiment, where a docking station is provided, one or more additional docking stations may be provided that communicate with a main docking station or gateway. Each docking station can accept an inhaler module or other healthcare related module and download compliance data. Each docking station can communicate its data with the main docking station by wire or wirelessly. The main docking station can collect the data from the other docking stations and transfer the data to a database located on a network to be used in a compliance reporting subsystem. In alternative embodiments, each docking station may individually communicate with the compliance reporting subsystem.

These and other objects, advantages and features of the invention will be more readily understood and appreciated by reference to the detailed description of the current embodiment and the drawings.

### BRIEF DESCRIPTION OF THE DRAWINGS

FIG. 1 is a front view of the current embodiment of the inhaler module and mounting element.

FIG. 2 is a side view of the current embodiment of the inhaler module and mounting element

FIG. 3 is a perspective exploded view of the current embodiment of the inhaler module and mounting element.

FIG. 4 is a first sectional view of the current embodiment of the inhaler module and mounting element.

FIG. 5 is a second sectional view of the current embodiment of the inhaler module and mounting element.

FIG. 6 is a perspective view of the first alternative embodiment of a mounting element mounted to one type of inhaler;

FIG. 7 is a perspective view of the first alternative embodiment of the mounting element mounted to another type of inhaler;

FIG. 8 is a perspective view of the first alternative embodiment of the mounting element mounted to yet another inhaler;

FIG. 9 is a perspective view of the first alternative embodiment of the mounting element mounted to still yet another inhaler;

FIG. 10 is a perspective view of the first alternative embodiment of the mounting element mounted to an even further inhaler;

FIG. 11 is a perspective view of the first alternative embodiment of the mounting element mounted to a medication container;

FIG. 12 is an exploded perspective view of the first alternative embodiment of the mounting element and an attachment element;

FIGS. 13-15 are exploded perspective views of the first alternative embodiment of the mounting element and portions of the inhaler module;

FIG. 16 is an exploded perspective view of the first alternative embodiment of the mounting element and the inhaler module;

FIG. 17 is a front exploded view of a second alternative embodiment of the mounting element and the inhaler module;

FIG. 18 is a rear exploded view of the second alternative embodiment of the mounting element and the inhaler module;

FIG. 19 is a perspective view of the second alternative embodiment of the mounting element adjacent internal components of the inhaler module;

FIG. 20 is a perspective view of the second alternative embodiment of the inhaler module with the mounting element removed;

FIG. 21 is a side and end view of a third alternative embodiment of mounting element mounting an inhaler module to an inhaler;

FIG. 22 is an exploded and disassembled side view of a third alternative embodiment of the mounting element, the inhaler module and the inhaler;

FIG. 23 is a perspective view of a third alternative embodiment of the mounting element with an inhaler shown in broken lines;

FIG. 24 is a side view of a third alternative embodiment of an inhaler module being installed in the mounting element;

FIG. 25 is a side view of a third alternative embodiment of an inhaler being installed in the mounting element;

FIG. 26 includes side view of examples of different inhalers having similar geometries;

FIG. 27 is an exploded side view of the inhaler module;

FIG. 28 includes sectional, side and front views of the assembled inhaler module;

FIG. 29 includes side views of fourth alternative embodiments of mounting elements mounted to the different inhalers of FIG. 26;

FIG. 30 is a block diagram illustrating the components of the inhaler module;

FIG. 31 is a top view of the current embodiment of the docking station;

FIG. 32 is a front view of the current embodiment of the docking station;

FIG. 33 is a perspective exploded view of the current embodiment of the docking station.

FIG. 34 includes front view of the inhaler module, mounting element and an inhaler being docked in a docking station;

FIG. 35 is a front view of the inhaler module being docked in a multi-port docking station therein;

FIG. 36 is a block diagram of an inhaler and related system

FIG. 37 is a block diagram illustrating the components of the docking station;

FIG. 38 is a flow chart illustrating an embodiment of the method of pairing an inhaler and inhaler module;

FIG. 39 is a flow chart illustrating an embodiment of the method of using the inhaler module and inhaler.

FIG. 40 is a flow chart illustrating another embodiment of the method of using the inhaler module and inhaler;

FIG. 41 is a representative view of a compliance report; and

FIG. 42 is a front view of three inhaler modules in communication with each other.

### DESCRIPTION OF EMBODIMENTS OF THE FIRST ASPECT

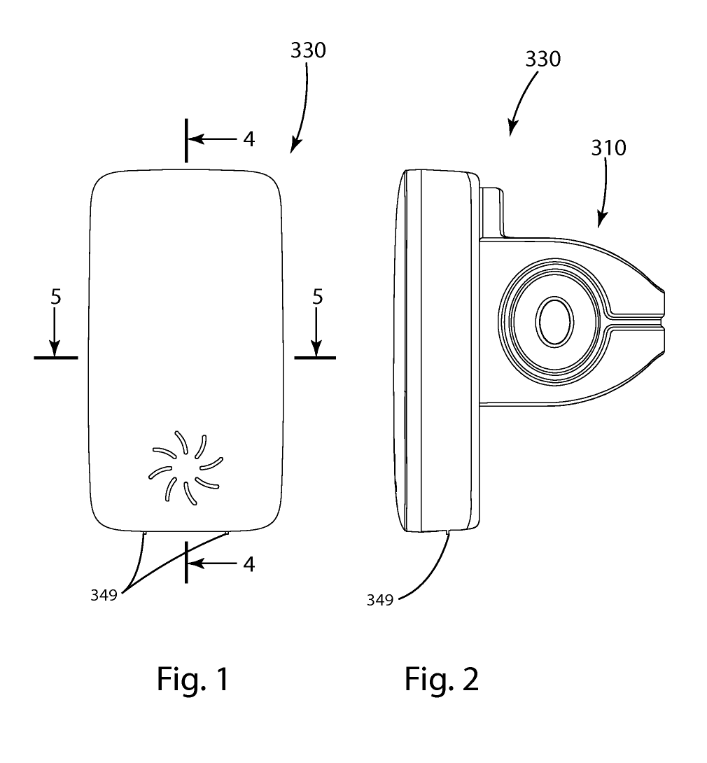

An apparatus of the current embodiment is shown in FIGS. 1-5 and generally designated 301. The current embodiment of the apparatus includes a mounting element 310, an inhaler module 330 and an attachment element 350.

The mounting element 310 generally mounts the inhaler module 330 to an inhaler. The mounting element 310 can generally expand from a relaxed mode to an expanded mode in order to accommodate a variety of different inhalers. The mounting element can include an elastic body 311. The elastic body 311 can be constructed from a variety of materials as will be discussed in more detail below. The elastic body can include apertures 398 and patterned recesses 399 to increase flexibility of the mounting element 310. In the current embodiment, the holes 398 are axially aligned through the center of the side walls 313 of the elastic body 311. The elastic body can be configured as a band having a front portion and a rear portion. As will be described in more detail later in connection with other embodiments, the elastic body 311 can define recesses 317, 319 that are configured along the upper edge and lower edge of the elastic body 311 respectively.

The elastic body and inhaler module are both removable with respect to the inhaler. That is, once the elastic body is installed on an inhaler, the inhaler module can be removed from the elastic body and then the elastic body can be stretched to remove it from the inhaler. Then, the elastic body can be stretched again to fit on another inhaler using the same process identified above in connection with the previous inhaler. In such a manner, the mounting element, and subsequently the inhaler module, can be used on multiple inhalers. Because inhaler modules can interchangeably fit in different mounting elements, other bands that fit other inhalers of different shapes and sizes can be utilized to mount an inhaler module.

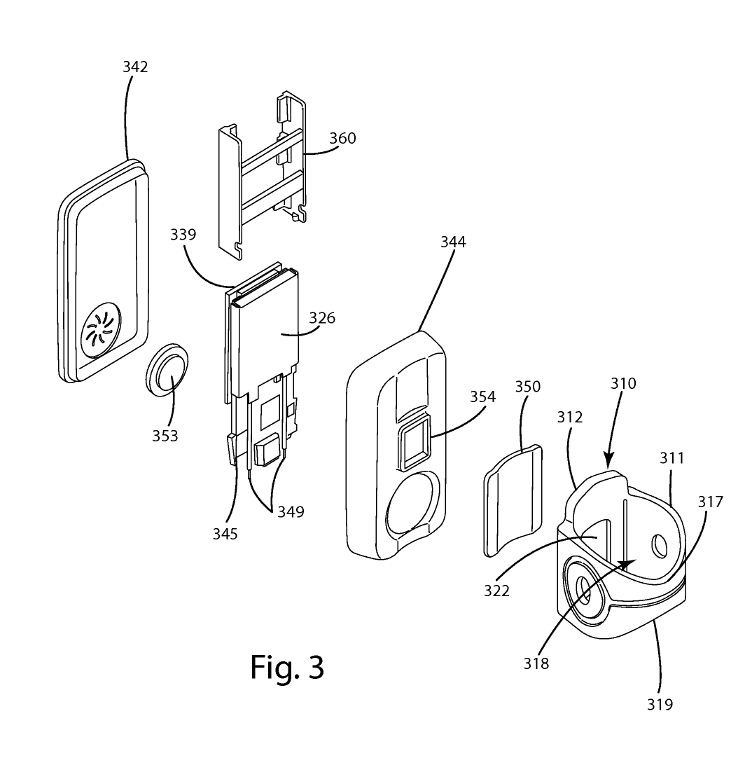

The elastic body 311 can be configured so that the band defines an internal hole or chamber 318 into which at least a portion of a respective inhaler is inserted. The elastic body 311 can also include a base 312. As shown in FIGS. 3-5, the base can be generally flat as compared to the remainder of the elastic body 111, which can be of a rounded or circular configuration to correspond to the exterior surfaces of most asthma inhalers. The base 312 can further define a recess or hole 327 that extends from the internal chamber 318 to the exterior of the mounting element 310. This hole 327 can be adapted to join the mounting element 310 with an attachment element 350, which further joins with the inhaler module 330.

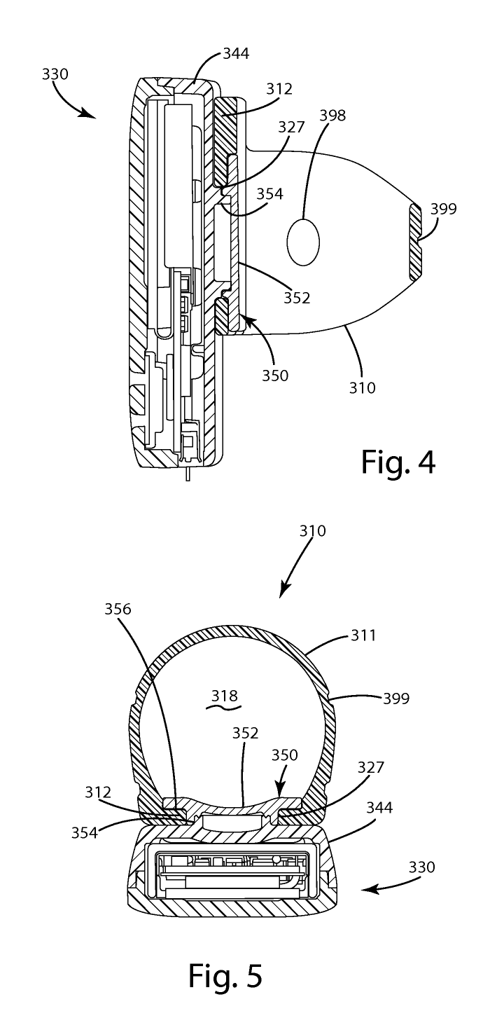

As shown in FIGS. 4-5, the attachment element 350 can include an attachment projection that defines a recess 356 between an attachment element base 354 and a flange member or “button” 352. The flange member 352 can be adapted to fit through the hole 327 when the periphery of the hole is stretched. After stretching and inserting the flanged element 352 into the hole 327, the periphery of the hole and the elastic body 311 can return to a relaxed or unstretched state (or perhaps a partially stretched state in some cases) so that it grips and/or generally surrounds the attachment element base 354. Generally, the periphery of the hole 327 can engage the base 354 and prevent rotation of the inhaler module 330 with respect to the mounting element 310.

The flange member 352 can be larger than the periphery of the hole so that the flange member 352 effectively captures a portion of the elastic body 311 between it and the base 354. Accordingly, the attachment element 350 and mounting element 310 can join with one another. Although not shown, the attachment element 350 alternatively can be integrally formed with the mounting element 310, and can include some type of fastener to join it with the inhaler module 130 or integrally formed with the inhaler module 330, and can include some type of fastener to join it with the mounting element 310.

The attachment element 350 can be integrally formed with the inhaler module 330. For example, in the current embodiment, the flange member 352 is joined with the attachment element base 354, which is integrally formed with the rear housing portion 344 of the inhaler module 330. The rear housing portion 344 of the inhaler module 130 can be sonic welded or otherwise joined with the attachment element 350.

Optionally, multiple different-sized mounting elements 310 can be provided in a kit with an inhaler module. In this kit, the mounting elements 310 can have different physical constructions, and can have different geometries, adapted to fit or grip or otherwise join the inhaler module with different types of inhalers. With such a kit, a user can select the appropriately dimensional mounting element 310, join it to the attachment element 350, and install the mounting element on the desired inhaler.

The current embodiment of the inhaler module does not include buttons or other tactile user input elements. Instead, the user can communicate with the inhaler module by 1) electrical communication through the dock (either wireless or through an electrical connection) or 2) by shaking the inhaler module to generate accelerometer output, which will both be discussed in more detail below.

Referring to FIG. 3, the components of an exemplary inhaler module will be described here. The inhaler module generally includes an outer housing separated into front and rear housing portions 342, 344. The housing portions can be injection molded parts constructed from a durable polymer. Optionally, the front and rear housing portions 342, 344 can include a seal that when joined together, the seal provides a semi or fully waterproof seal between the housing portions. The housing portions 342, 344 can be configured to form an internal cavity within which a printed circuit board 345, display assembly 339, battery 326, and speaker 345 interfit. Optionally, the printed circuit board 345, display assembly 339, and battery 326 can be mounted to a chassis 360 that can be installed within the internal cavity. In alternative embodiments additional, fewer or different components can be mounted interfit in the internal cavity between the housing portions. The printed circuit board 345 can be electrically connected a pair of externally exposed contacts 349, which can be used to interface to a docking station.

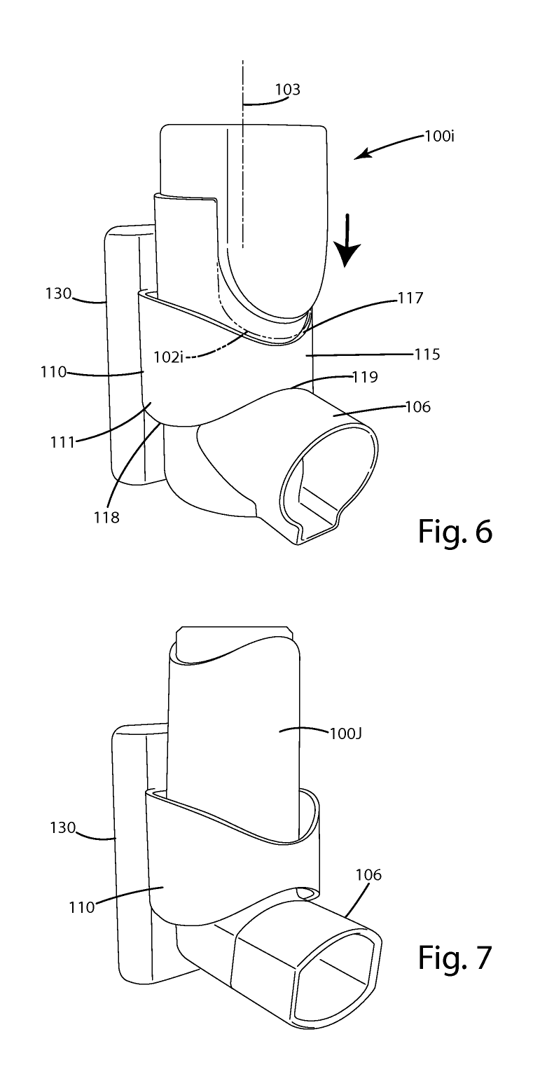

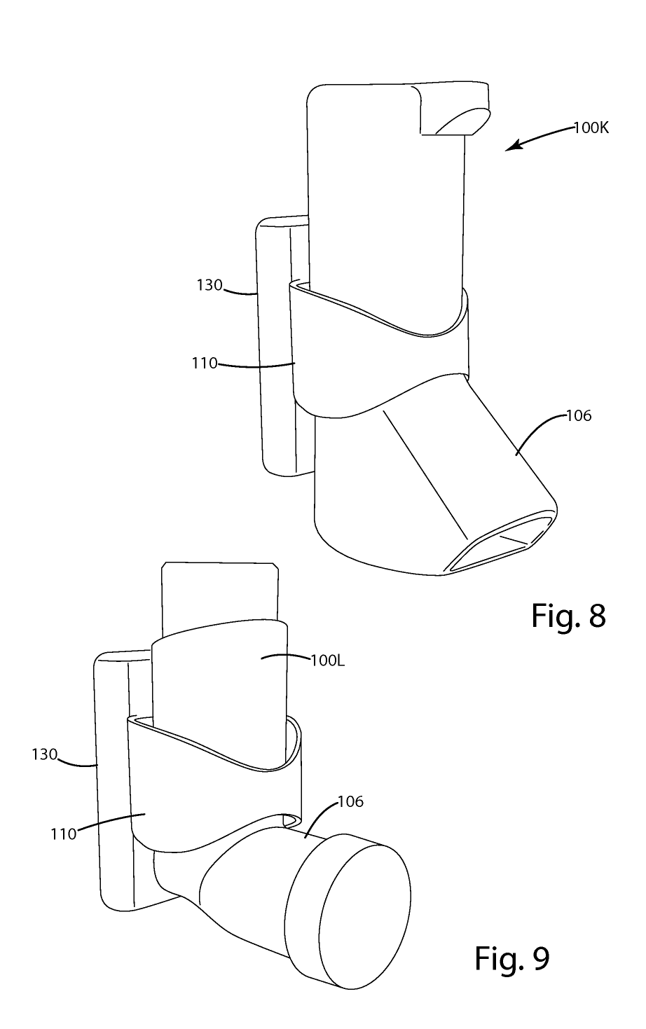

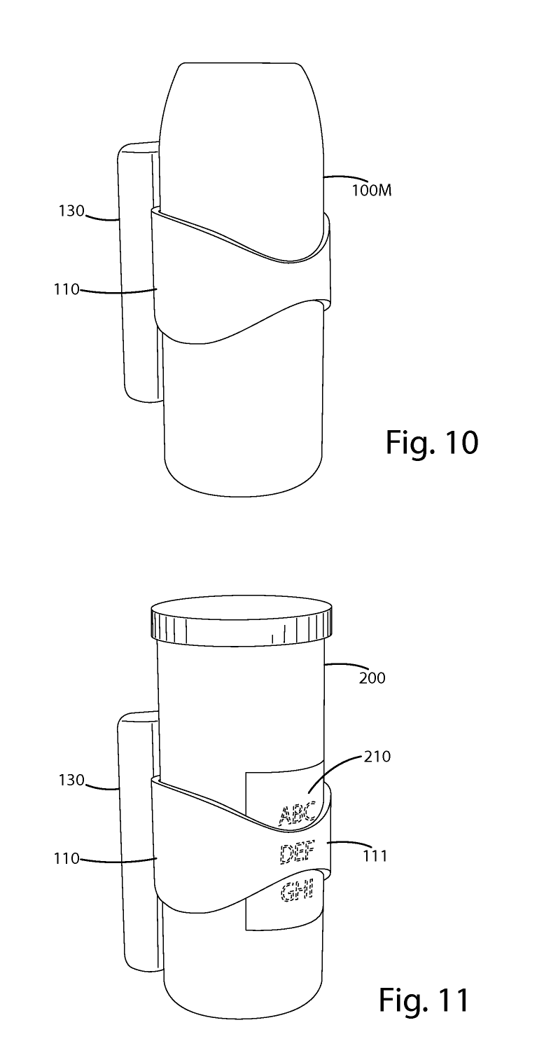

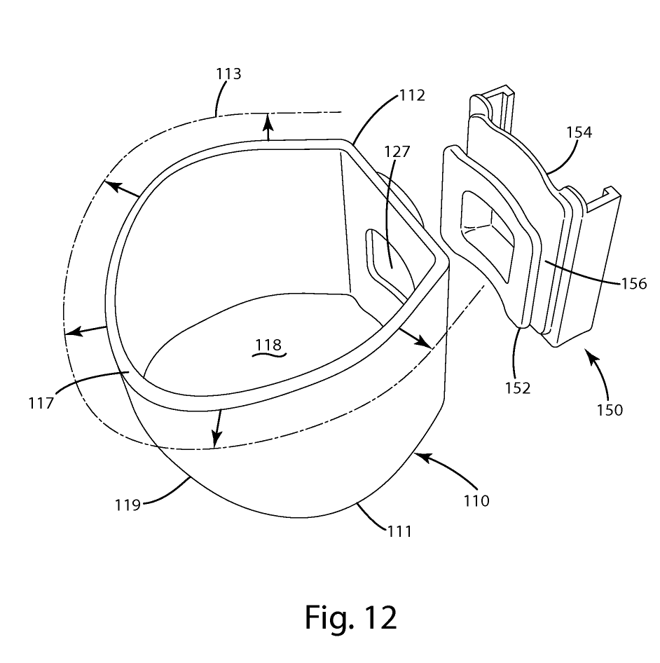

A first alternative embodiment of the first aspect is illustrated in FIGS. 6-16. This embodiment of the mounting element is depicted as being attached to a variety of different inhalers 100i-100m as illustrated in FIGS. 6-10 and generally designated 110. Although only one embodiment of the mounting element is shown attached to the different inhalers, other embodiments can also be attached to a variety of different inhalers in a similar fashion. The embodiment of the mounting element depicted in FIGS. 6-16 is similar to the mounting element of the current embodiment above with several exceptions. For example, like the above-described mounting element, the mounting element 110 can generally expand from a relaxed mode to an expanded mode as shown by the arrows and broken lines in FIG. 12. In the expanded mode, the mounting element 110 can wrap around at least a portion of the inhaler, grip the inhaler 100, and/or hold the inhaler module 130 in a predetermined configuration or orientation relative to the inhaler.

As shown in FIGS. 6 and 12, the mounting element can include an elastic body 111. The elastic body 111 can be constructed from any of the materials explained above in connection with the other embodiments described herein. The elastic body can be configured as a band having a front portion and a rear portion. In the front portion, the elastic body 111 can define a recess 119 that is configured along the lower edge 118 of the elastic body 111. This recess 119 can be dimensioned and sized so that the second portion or mouthpiece 106 of the inhaler 100 generally fits at least partially within the recess. The recess 119 can be of a geometric configuration that corresponds to the mouthpiece or second portion 106. It also may be configured so that the lower edge 118 in the portion of the recess 119 engages the second portion or mouthpiece 106. With such engagement, the elastic body 111 can prevent or impair an inadvertent or accidental rotation of the inhaler module 130 about the longitudinal axis 103 of the inhaler. Accordingly, a user can consistently grasp the inhaler and/or manipulate the inhaler.

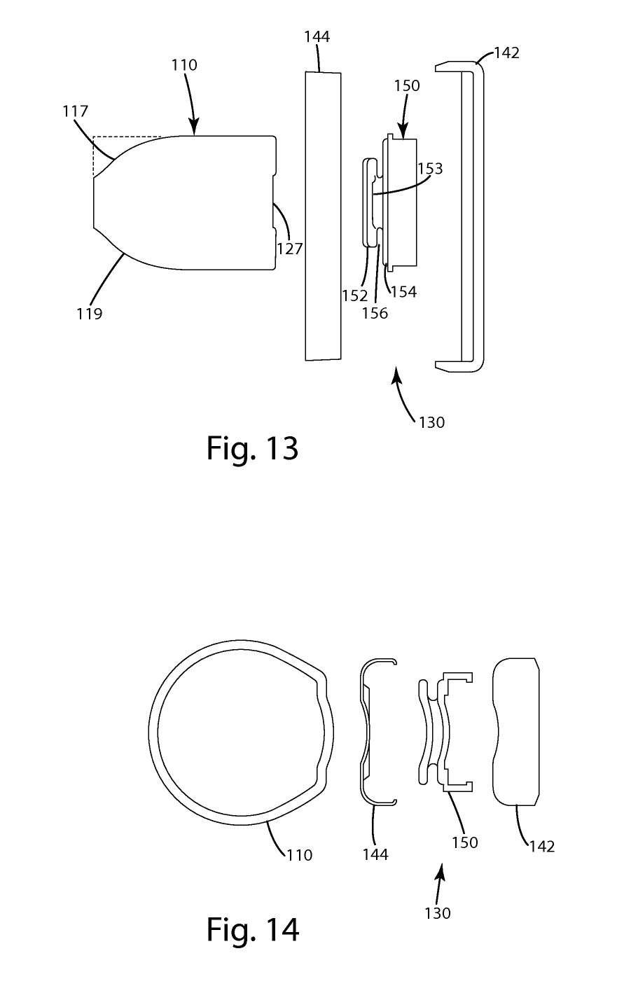

The front portion of the mounting element 110 optionally can define a recess 117 in the upper edge of the elastic body 111. This recess can be generally symmetric to the first recess 119. The configuration of the second recess 111 can be varied and of a different geometric configuration and/or dimension. As shown in FIG. 6, the second recess 117 can be configured so that the upper edge of the elastic body 111 does not interfere with the downward movement of the movable element 102i, as shown with the arrow and in phantom lines. Accordingly, the movable element 102i of the inhaler is free to move so that administration of a particular dose of medication from the inhaler is not affected by the mounting element 110. Further optionally, where an inhaler does not include a movable element, like the inhalers shown in FIGS. 7-9, the upper edge of the mounting element 110 can be without a second recess 111. As an example, FIG. 13 illustrates a construction including an upper edge in broken lines with the upper or second recess 117 absent from the mounting element 110.

Returning to FIGS. 6 and 12, the elastic body 111 can be configured so that the band defines an internal hole or chamber 118 into which at least a portion of a respective inhaler is inserted. The elastic body 111 can also include a base 112. As shown in FIGS. 12-15, the base can be generally flat as compared to the remainder of the elastic body 111, which can be of a rounded or circular configuration to correspond to the exterior surfaces of most asthma inhalers. The base 112 can further define a recess or hole 127 that extends from the internal chamber 118 to the exterior of the mounting element 110. This hole 127 can be adapted to join the mounting element 110 with an attachment element 150, which further joins with the inhaler module 130.

As shown in FIGS. 12-15, the attachment element 150 can include an attachment projection 152. The attachment projection 152 can define a recess 156 between an attachment element base 154 and a flange member or “button” 152. The flange member 152 can be adapted to fit through the hole 127 when the periphery of the hole is stretched. After stretching and insertion of the flanged element 152 into the hole 127, the periphery of the hole and the elastic body 111 can return to a relaxed or unstretched state (or perhaps a partially stretched state in some cases) so that it grips and/or generally surrounds the button base 153 (FIG. 13). Generally, the periphery of the hole 127 can engage the base 153 and prevent rotation of the base 153 within the hole 127.

The flange member or button 152 can be larger than the periphery of the hole so that the button 152 effectively captures a portion of the mounting element of the elastic body 111 between it and the attachment element base 154. Accordingly, the attachment element 150 and mounting element 110 can join with one another. Although not shown, the attachment element 150 alternatively can be integrally formed with the mounting element 110, and can include some type of fastener to join it with the inhaler module 130. For example, the attachment element base 154 can be integrally molded with the base 112. In such a construction, the hole 127 and the button 152 can be absent.

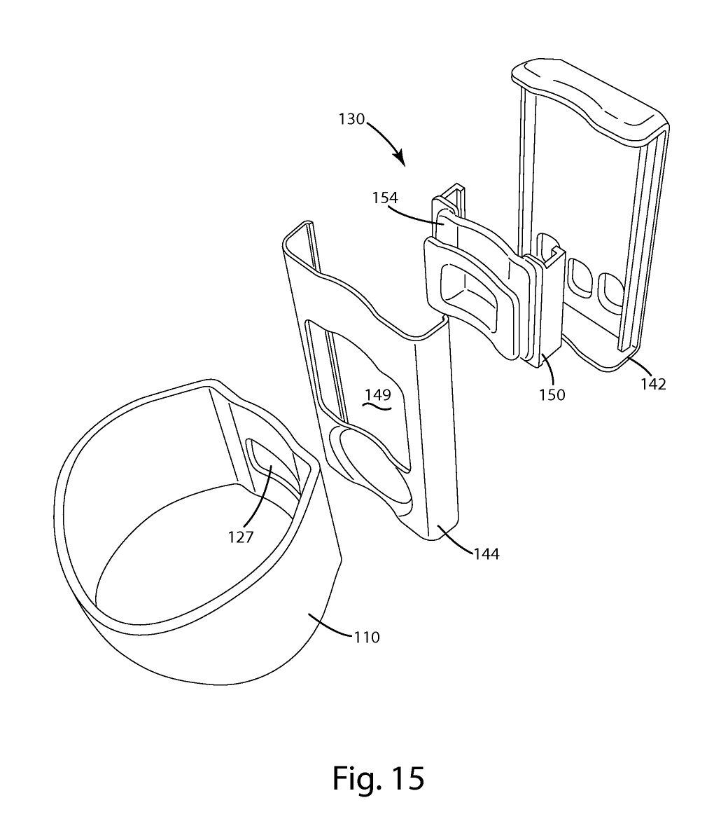

The attachment element 150 can be configured to join with the housing portions of the inhaler module 130. For example, as shown in FIGS. 14 and 15, the rear housing portion 144 can define an aperture 149 through which a portion of the attachment element 150 fits. Optionally, the base 154 of the attachment element 150 can be configured to be the same dimension and size as the outer periphery of the housing aperture 149 so that the base effectively closes and fills the aperture when the attachment element 150 is placed adjacent the rear housing portion 144. Of course, the attachment element 150 can be integrally formed with the rear housing element or another portion of the housing for the inhaler module 130 if desired.

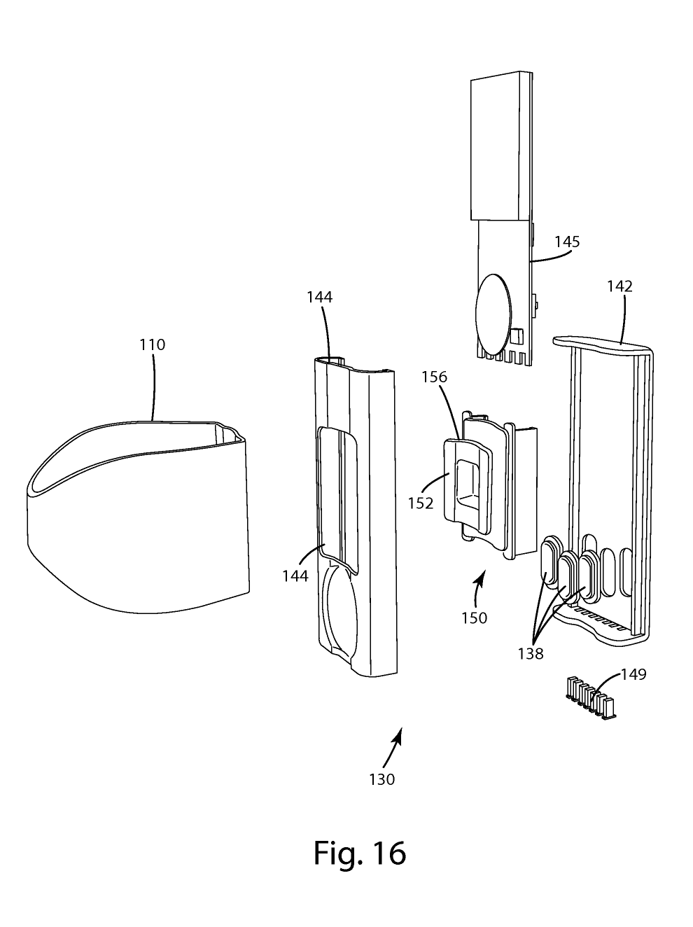

In general, a portion of the attachment element 150 can be housed within the housing portions 142 and 144, with a remaining portion, for example the button 152 projecting rearwardly from the housing so that it can engage the mounting element appropriately. As shown in FIG. 16, the other internal components, such as the circuit board 145, module buttons 138, and connectors 149 can be at least partially housed within the front 142 and rear 144 housing portions as with the embodiments described above.

As with the current embodiment, multiple different-sized mounting elements 110 can optionally be provided in a kit with an inhaler module. In this kit, the mounting elements 110 can have different physical constructions, and can have different geometries, adapted to fit or grip or otherwise join the inhaler module with different types of inhalers. With such a kit, a user can select the appropriately dimensional mounting element 110, join it to the attachment element 150, and install the mounting element on the desired inhaler.

The mounting element and module described herein provide opportunities to use these components in other applications. For example, as shown in FIG. 11, the mounting element 110, and in particular, the elastic body 111 can be positioned around the exterior of a container 200 and can mount the module 130 to that container. This container 200 can be a medication container containing pills, tablets, capsules or other medical or treatment items.

To install the mounting element 110 and module 130, the elastic body 111 can be stretched to an expanded mode, applied over an exterior surface of the container 200 and then allowed to relax somewhat so that the mounting element 110 grips the exterior surface of the container 200. In this manner, the module 130 can be joined with the container 200.

The elastic body 111 can be constructed from a transparent material that enables a user to read indicia 210 associated with the container 200 through the elastic body 111. For example, where the indicia 210 includes instructions for taking medication, such as pills contained in the container, a user can review those instructions, viewing them through the elastic body 111. Optionally, the elastic body 111 can be opaque, but configured so that it does not obscure with a user's view of indicia 210 associated with the container. For example, the elastic body 111 can include a hole or recess in the region where the device 210 is located to facilitate viewing of the indicia.

The module 130 used in applications, such as that shown in FIG. 11, where it is mounted on a medication container, can be programmed slightly differently from that of the embodiments described above. For example, the module 130 can be programmed to alert a user to take a medication from the container at a particular time, and can be programmed to query the user to input information about their general health or well-being before and/or after taking the medication. Optionally, where an accelerometer is included in the module 130, the accelerometer may react to different motions appropriate to the use of that medication. The output queries can be specifically tailored to the particular medication, in contrast to queries generally tailored to administering a dose from an inhaler.

The information collected by the module and/or input to the module by a user regarding the administration of the medication, and/or other health-related information, can be stored in the module 130 and later uploaded to a docking station as described in the embodiments above. In cases where a user utilizes multiple medications and/or inhalers, multiple modules can be associated with the related medication containers and/or inhalers. Data collected from respective modules can be uploaded to a docking station, such as those described herein. That data can further be transferred to a physician or other healthcare provider or entity using any of the techniques described herein. A third party can then use all or part of the information collected by the different modules to assess the patient's treatment and health in general. Based on that assessment, the physician can modify treatment or identify trends or risk factors and take appropriate action.

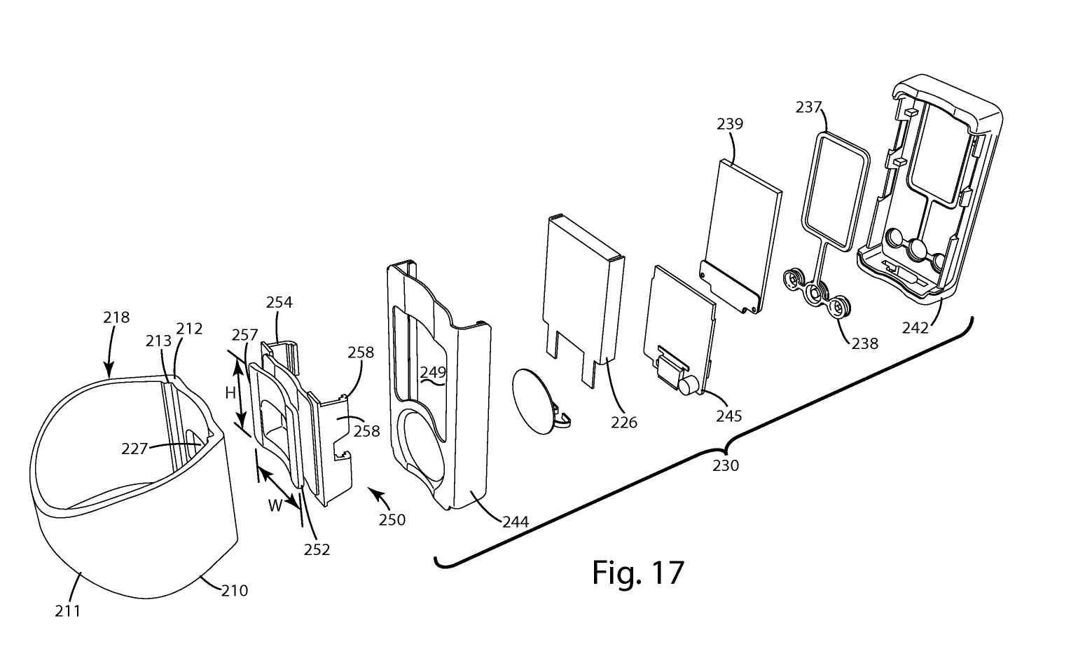

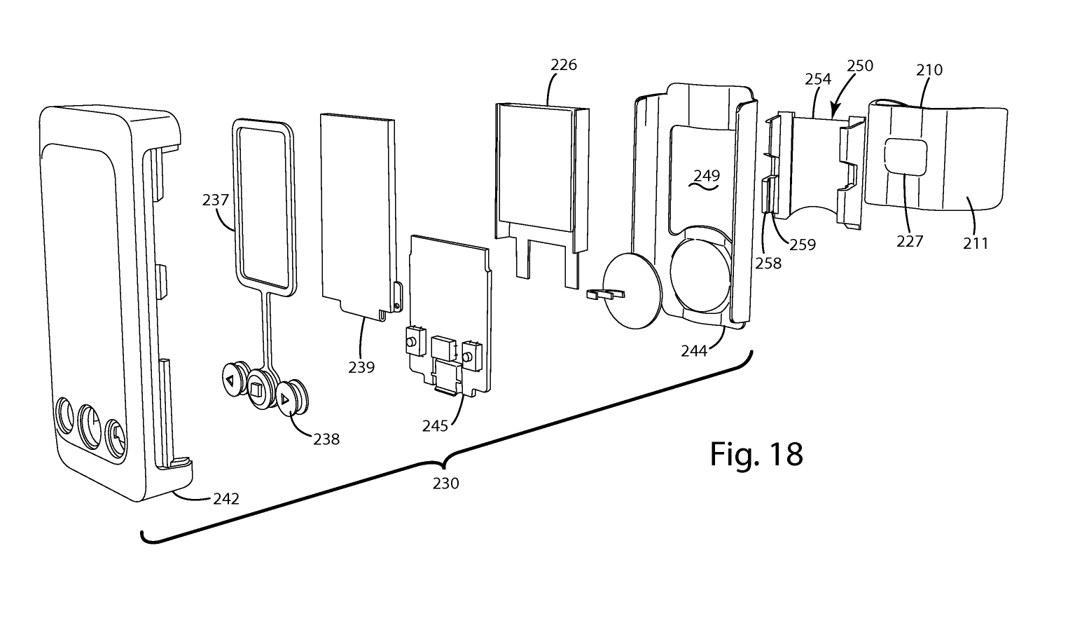

A second alternative embodiment of the mounting element and inhaler module is illustrated in FIGS. 17-20. This embodiment of the mounting element 210 and the inhaler module 230 is similar to the mounting element and inhaler modules of the embodiments above with several exceptions. For example, connection between the attachment element 250 and the body 211 can be configured to prevent or impair inadvertent rotation of the inhaler module 230. The body 211 can include a groove having peripheral edges 213 defined in the base 212. The attachment element 250 can include a flange member or button 252, which like the embodiments above, fits through the hole 127 defined by the elastic body 211. The button can have a height H that is optionally greater than the width W. Alternatively, the height H can simply be great enough so that when the flange member or button 252 is inserted through the hole 227, the height H prevents the attachment element 250 from rotating relative to the mounting element 210. Further optionally, the side edges 257 of the button 252 can engage the periphery of the groove 213 to further reduce or impair twisting or rotation of the attachment element 250, and thus the inhaler module 230 relative to the mounting element 210. In turn, this can prevent inadvertent rotation or disruption of the inhaler module relative to the inhaler to which the mounting element 210 is attached. This can provide a consistent structure for the user to grasp, and also can prevent or impair the inhaler module 230 from being inadvertently disconnected from the elastic body 211.

The attachment element 252 also can fit through an aperture 249 of the rear housing 244 as described in some of the embodiments above. The front housing element 242 can join with the rear housing element 244 to house the various components including the battery 226, the PCB or circuit board 245, the display 239 and the button elements 238.

As shown in FIGS. 17-20, the button element 238 can include an extended buffer portion 237 which is adapted to be placed between the display 239 and the outer housing 242. This buffer portion 237 can be constructed from the same overmolded elastomeric or other material as the buttons 238. Accordingly, it can provide cushioning and impact absorption to generally protect the display, particularly in situations where the display may be forced toward the housing 242 upon the module 230 being dropped.

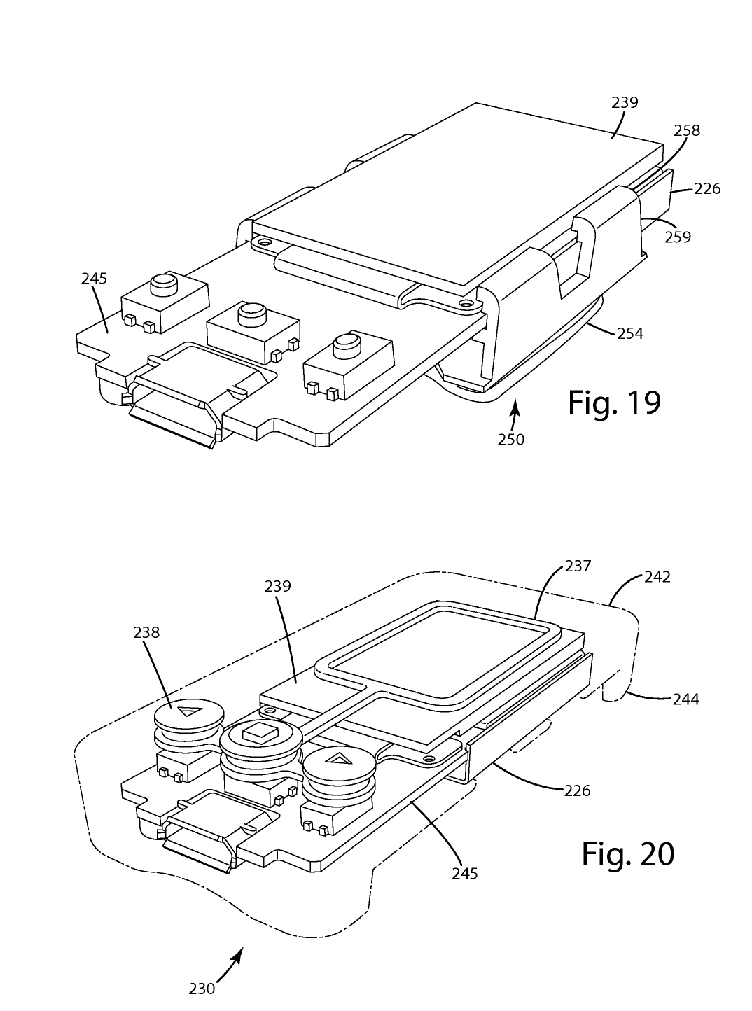

As shown in FIG. 19, the attachment element 250 can also be configured to act as a chassis to hold the PCB or circuit board 245, the battery 226 and/or the display 239. For example, the attachment element 250 can include arms 259 that extend rearwardly from the base 254. These arms can terminate at ends 258 which may be of a hooked, curved or angled configuration. The ends 258 in such a configuration can engage the display 239 and/or the battery or PCB 245 to generally capture one or more of those components between those ends and the base 254. This can prevent or impair movement of these components within the inhaler module housing elements 242 and 244. Various embodiments of the inhaler module will be discussed in detail later in connection with other aspects.

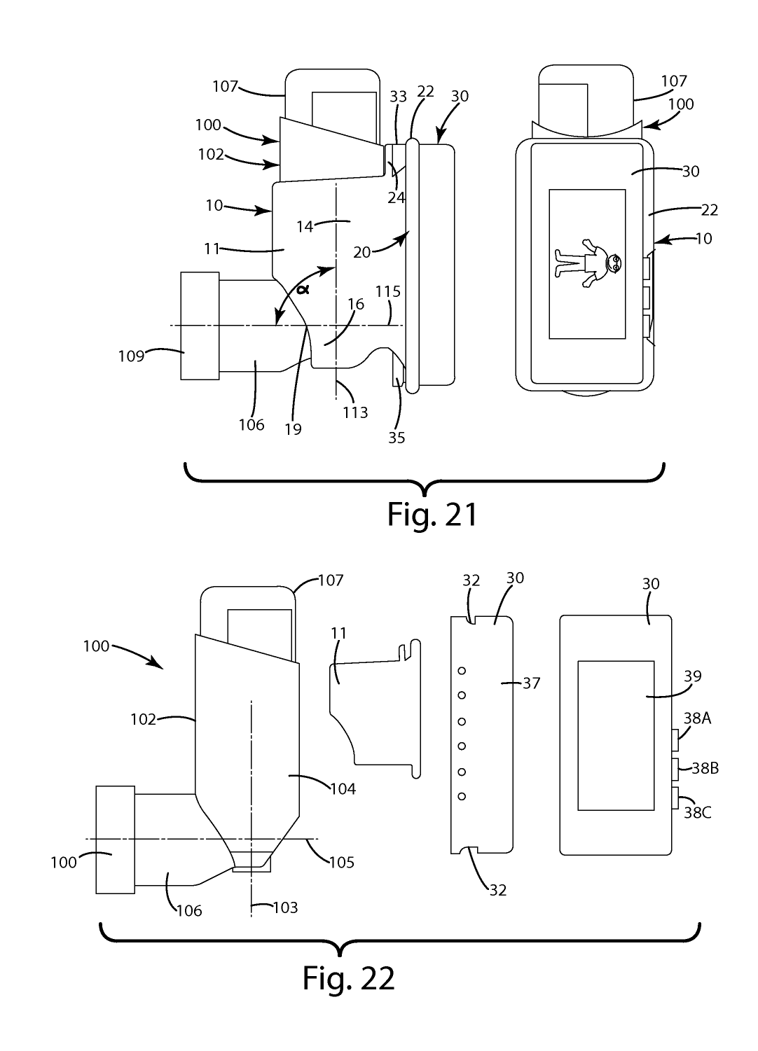

A mounting element of the third alternative embodiment of the first aspect is shown in FIGS. 21-28 and generally designated 10. The mounting element 10 generally mounts the inhaler module 30 to the inhaler 100. As illustrated, the inhaler can be a conventional metered dose inhaler which can include a housing 102 that is adapted to receive a canister of medication 107. For example, as shown in FIG. 22, the housing 102 can include a first portion 104 and a second portion 106. The first portion can be configured to receive the canister 107 of asthma medication.

Generally, the first portion 104 of the inhaler acts as a gripping portion while a user depresses the canister 107 to administer medication. The second portion 106 can be configured to form a mouthpiece which includes an opening 109 through which medication from the canister 107 is dispensed into the mouth of a patient or subject. In some embodiments, the inhaler module can attach to the cap of the inhaler.

The first portion 102 of the inhaler 100 can be aligned with a vertical inhaler axis 103. The second portion 106 of the inhaler can be aligned with a generally horizontal inhaler of axis 105. The first and second axes 103 and 105 can be transverse to one another. For example, they can be at approximately 90°, 120°, 140° or other angles relative to one another. Several examples of conventional metered dose inhalers having similar geometries, where the first portion of the inhaler generally holds a canister, and a second portion which forms a mouthpiece through which a spray is administered, are illustrated in FIG. 26.

While contemplated for use with the conventional metered dose inhalers in FIGS. 22 and 26, the mounting element 10, inhaler module 30 and docking station 70 (described below) contemplated herein also can be modified to be used with inhalers including a spacer, nebulizers, as well as dry powder inhalers, such as those used for the administration of medication sold under the trade name ADVAIR. Further, the mounting element 10, inhaler module 3, and docking station 30 also can be used across a variety of categories of asthma medicines and delivery systems, for example, inhaled bronchial dilators, inhaled anti-inflammatory systems and combination medicines. Inhaled bronchial dilators can sometimes be referred to as resting inhalers that relax tight airways and help deal with coughing, wheezing and shortness of breath. Inhaled anti-inflammatory systems, which are typically considered maintenance inhalers, can use steroids and other anti-inflammatories to reduce and prevent airway inflammation and swelling. Such inhalers are typically used on a daily basis under a compliance regimen to prevent acute symptoms of asthma. Other combination devices may use bronchodilators and costeriod components. Again, although discussed primarily in conjunction with metered dose maintenance inhalers and rescue inhalers, the components described herein can be used across a wide variety of other types of inhalers, nebulizers and asthma medication administration devices.

Returning to FIGS. 21 and 22, the first portion 14 of the elastic body is adapted to be joined in close proximity to the first portion 104 of the housing 102. The second portion 16 of the elastic body 11 is adapted to be mounted in close proximity to the second portion or mouthpiece 106 of the inhaler. In general, the second portion 16 can define an opening 19 through which the mouthpiece 106 projects. The opening 19 can then be configured transverse to the longitudinal axis 113 of the mounting element 10. The opening 19 can be aligned with the transverse axis 115 of the mounting element 10 as shown in FIG. 22. Although shown generally at a right angle, the longitudinal axis 113 and transverse axis 115, and the respective first and second mounting element portions 14, 16, can be at virtually any angle a relative to one another. For example, angle α can be 90°, 100°, 110°, 120°, 130°, 140°, 150° or more, or less, depending on the configurations of the inhaler to which the mounting element is joined.

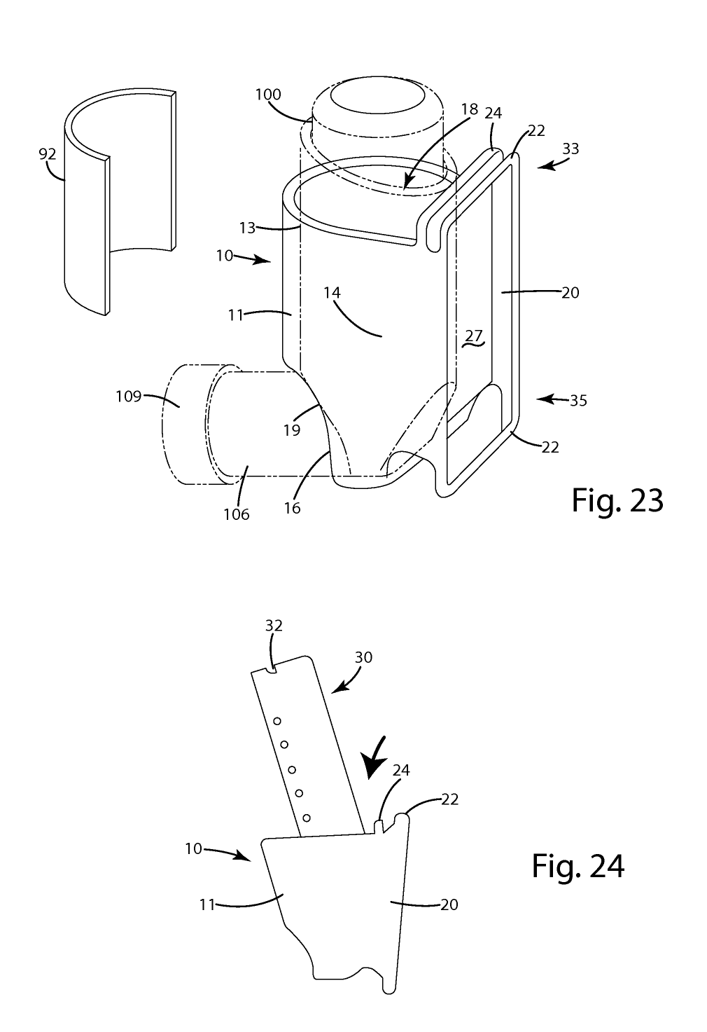

With reference to FIGS. 21 and 23, the mounting element can also define an internal cavity 18. Internal cavity 18 can be of a pre-determined dimension which is slightly smaller than the external dimensions of a target inhaler to which the mounting element 10 is to be mounted. Optionally, the internal cavity can be of dimensions that are smaller than the smallest dimensions in a given set of inhalers to which the mounting element is to be mounted. For example, the mounting element 10 can be designed to be joined with the eight conventional inhalers illustrated in FIG. 26. In this case, the internal cavity 18 of the mounting element can be dimensioned so that when the mounting element 10 is placed over the inhaler having the smallest dimensions of the eight inhalers 100a-100h, the body 11 of the inhaler still sufficiently engages at least a portion of the outer dimensions of that smaller inhaler to hold the mounting element in a fixed position and orientation relative to that smallest inhaler. The elastic body 11 can be also configured so that when stretched or expanded over that smallest inhaler, it still sufficiently grips the inhaler to hold it in that fixed position and orientation. Likewise, the elastic body 11 can be configured so that it can be stretched to accommodate the largest inhaler having the largest dimensions of the selected group of inhalers to which the mounting element is to be mounted.

To accommodate a wide variety of dimensions across different inhalers, the elasticity of the elastic body 10 can be modified in a variety of ways. For example, the walls of the elastic body 11 can be increased or decreased in thickness to increase elasticity or stretchability. Alternatively, or in combination, the material from which the elastic body is constructed can vary. For example, a softer, more elastic rubber can be replaced with a harder, less elastic rubber when less elasticity and/or less stretching is desired. Alternatively, or in combination, the elasticity of the elastic body 11 or certain portions of it can be varied by including apertures in locations where increased stretching of the body is desired.

The elastic body 11 and its components can be constructed from a wide variety of materials. For example, in the current embodiment, the elastic body 11 can be constructed from an elastomer, that is, a polymer, having a low Young's modulus and high yield strain when compared with other materials. Suitable examples of elastomers for use with the current embodiment include natural rubber, synthetic rubber, polybutadiene, nitrile rubber, ethylene propylene rubber, ethylene propylene diene rubber, polyacrylic rubber, silicone rubber, ethylene-vinyl acetate, thermoplastic elastomers, thermoplastic polyurethane, thermoplastic olefins and other materials having comparable elastic properties, so they can be stretched over an inhaler or similar device and subsequently provide a gripping force to hold the mounting element in a fixed orientation or position relative to the inhaler. Optionally, the elastomer can be of a durable construction so that can withstand at least six months to a year of normal use on one or more inhalers, and can be transferred from one inhaler to another one or more times during that time frame.

In some cases, it is contemplated that the mounting element 10 may not precisely fit a particular inhaler. In which case, shims may be provided with the mounting element. An example of a suitable shim is a small piece of material 92 (FIG. 23) that fits between the inhaler 100 and a portion of the internal cavity 18 of the mounting element 10. The shim can be configured of a desired thickness, width and length so that the desired forces are exerted by the mounting element on the inhaler to hold the mounting element 10, and subsequently the inhaler module 30, in a desired orientation and position relative to the inhaler 100.

Returning to FIGS. 21-24, the mounting element 10, and particularly the elastic body 11 can include an attachment element 20. This attachment element 20 can be joined to the first portion 14 of the elastic body, projecting rearwardly, away from the mouthpiece 106 of the inhaler 100 when the mounting element 10 is installed on the inhaler 100. The attachment element 20 can include one or more fasteners that join the inhaler module 30 to the mounting element 10, and subsequently to the inhaler 100. As illustrated in FIGS. 24-26, the attachment element 20 can include bands 22 that wrap around the ends of the inhaler module 30. Optionally, the inhaler module 30 can include inhaler module grooves 32, which are defined around a portion of the housing 34 of the inhaler module 30, to accept the bands 22. Although the mounting element 10 as illustrated includes the peripheral bands 22, it can include other fasteners or features that interlock or otherwise join with the inhaler module 30 to hold the inhaler module 30 in a fixed position relative to the inhaler 100 after installation.

As illustrated in FIG. 23, the mounting element 10 also can include a mounting base 24 which optionally can separate the attachment element 20 from the internal cavity 18 of the elastic body 11. This mounting base 24 can simply be a divider or a wall that separates the cavity 18 from the recess or capturing area 27 within which the inhaler module 30 is generally positioned. The base 24 can extend from a position corresponding to the upper portion 33 of the inhaler module as shown in FIG. 24 to the lower portion of the inhaler module 35 as shown in FIGS. 21 and 23. Alternatively, the mounting base 24 can simply extend along a portion mounting element 10 so that it separates the inhaler module 30 from contact with the inhaler 100. In this manner, excessive vibration or damage or impact between these two elements can be minimized. Of course, if desired, the mounting base 24 can be entirely absent from the mounting element 10 or replaced with another suitable feature.

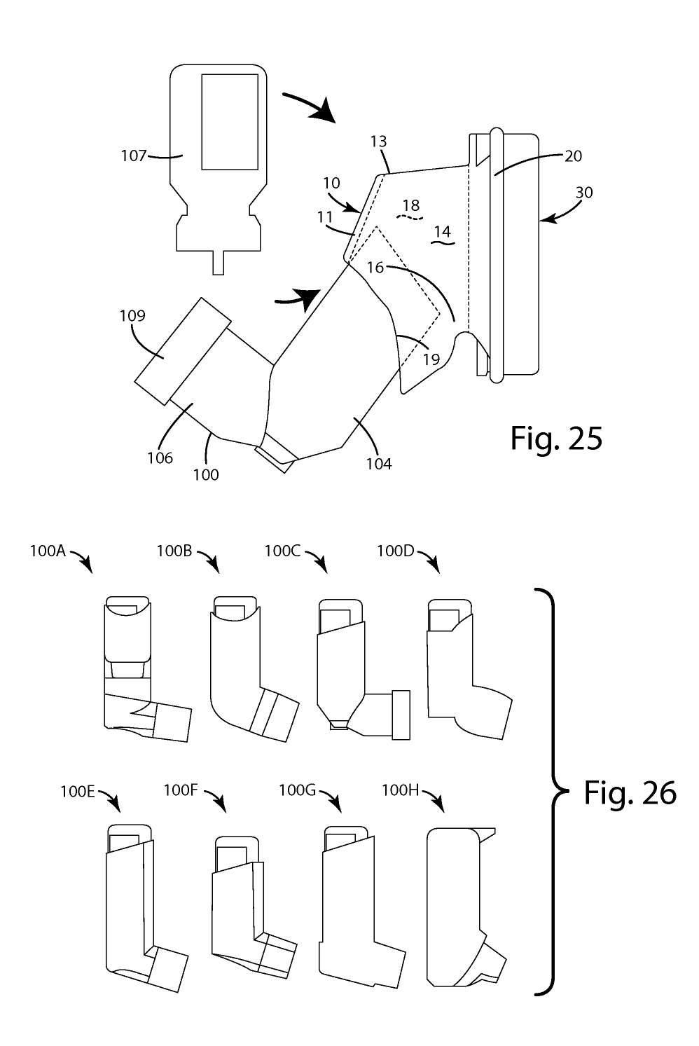

The current embodiment also can provide a method of using the mounting element to install an inhaler module 30 on an inhaler 100. With reference to FIGS. 24-25, this method will be briefly described here. In one step, the inhaler module 30 can be inserted in the elastic body 11 of the mounting element 10 as shown in FIG. 24. The inhaler module 30 is pushed downward into the elastic body 11 sufficiently so that it engages the attachment element 20. The bands 22 of the attachment element can be aligned with the grooves 32 of the inhaler module 30 to sufficiently position the inhaler module 30 relative to the mounting element 10.

Given the elasticity of the mounting element 10, the portions of the elastic body 11 will expand or stretch so that the components can come into close proximity to portions of the inhaler module 30. For example, the bands 22 can stretch 1% to 25% or more to fit around the inhaler module and within the grooves 32. Although the inhaler module 30 is illustrated as being inserted within the internal cavity 18 of the elastic body 11, and then into the engagement with attachment element 20, the inhaler module 30 can be inserted into the attachment element 20 externally, rather than through the internal chamber 18. In which case, the inhaler module can be inserted into the recess 27, with the bands 22 wrapped around the ends of the inhaler module 30.

Returning to FIG. 25, in another step, the inhaler 100 is joined with the mounting element 10. Generally, the inhaler 100 can be inserted so that the first portion 104 enters the opening defined in the second portion 16 of the elastic body 11. Subsequently, the first portion 104 enters the internal cavity 18 of the mounting element 10. The first portion 104 is pushed into the cavity until the upper portion of the inhaler protrudes through the top opening 13 of the mounting element 10. In this configuration, the mounting element does not interface with or even contact the canister 107 joined with the inhaler. With the inhaler 100 installed in the mounting element 30, the mouthpiece 106 of the inhaler 100 generally protrudes through the opening 19 as illustrated in FIG. 24. In another step, the canister 107 can be inserted into the inhaler 100.

After the mounting element is installed on the inhaler 100, the inhaler module 30 can be reconfigured, re-oriented or otherwise rotated about the body of the first portion 104 or the second portion 106 so that it is in a desired orientation relative to these components. In general, at this point, the mounting element 10 mounts the inhaler module 30 piggyback to the inhaler 100. When fully installed or appropriately positioned, the mounting element 10 and inhaler module optionally do not affect the airflow into or out from the inhaler, nor do these elements prevent or delay the use of the inhaler, nor do these elements otherwise obstruct moving parts associated with the inhaler or the canister, even while the inhaler is being grasped by a user to administer medication.

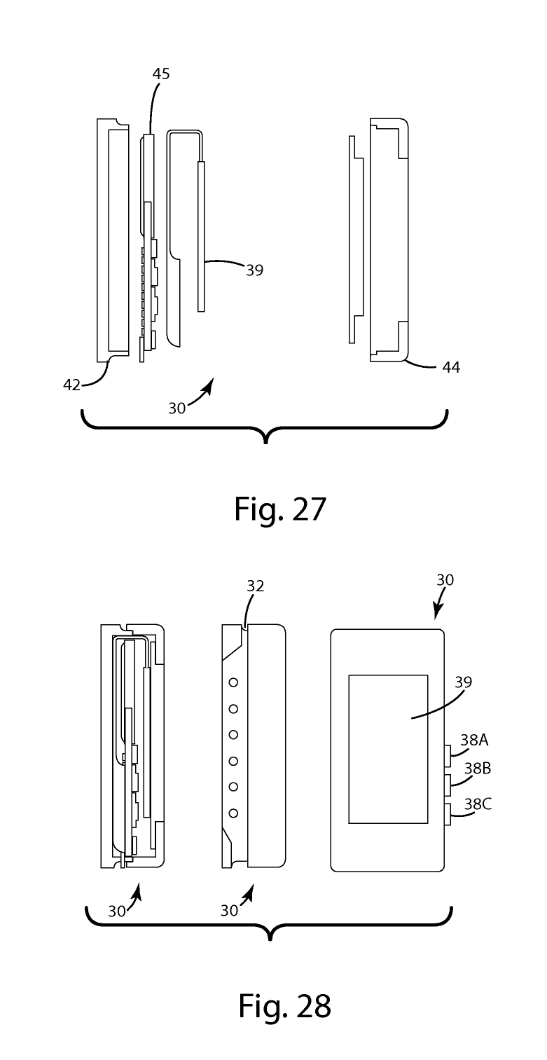

As shown in FIGS. 27-28, the components of an exemplary inhaler module will be described here. The inhaler module generally includes an outer housing separated into front and rear housing portions 42, 44. The housing portions can be injection molded parts constructed from a durable polymer. Optionally, the front and rear housing portions 42, 44 can include a seal that when joined together, the seal provides a semi or fully waterproof seal between the housing portions. The housing portions 42, 44 can be configured to form an internal cavity within which a printed circuit board 45 and display assembly 39 interfit. The housing can also define apertures through which buttons or contact points 38a-c extend.

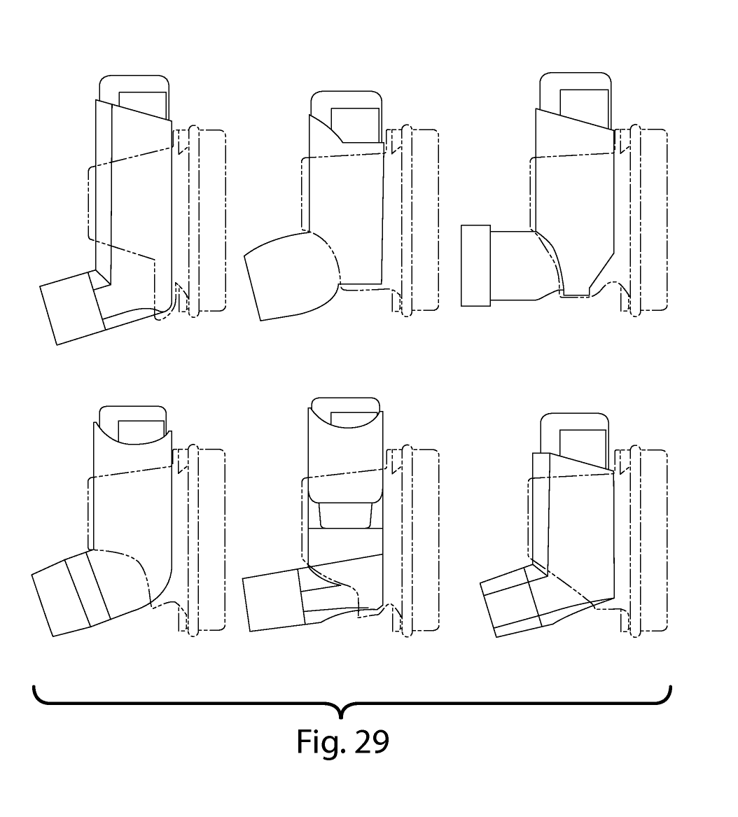

Referring to FIG. 29, several additional alternative constructions of the mounting element are illustrated. In general, all of these constructions include a first portion structure or band that wraps around at least the vertical portion of the inhalers. In these embodiments, like the current embodiment, the mounting element is configured so that it exerts a slight compressive holding force against exterior portions of the inhaler, thereby holding the inhaler modules in a pre-determined spatial configuration relative to the inhaler.

The various embodiments of inhaler modules and mounting elements described herein are exemplary. To the extent possible, each of the described embodiments can operate and function in a manner similar to any of the other embodiments, or as otherwise desired for a particular application. Further, to the extent appropriate, features from one embodiment can be incorporated into a different embodiment. For example, to name a few, any of the inhaler modules can be constructed without buttons or other tactile user input, or, any of the mounting elements can be constructed with holes 398 in the side walls in order to increase flexibility of the elastic band.

### DESCRIPTION OF EMBODIMENTS OF THE SECOND ASPECT

The second aspect relates generally to indicating, at a glance, levels of compliance with a medication treatment regimen to a user or caregiver. The current embodiment of a compliance at a glance system is described below. To facilitate the description of the compliance at a glance system, some embodiments of exemplary inhaler modules and some embodiments of exemplary docking stations will be described in detail.

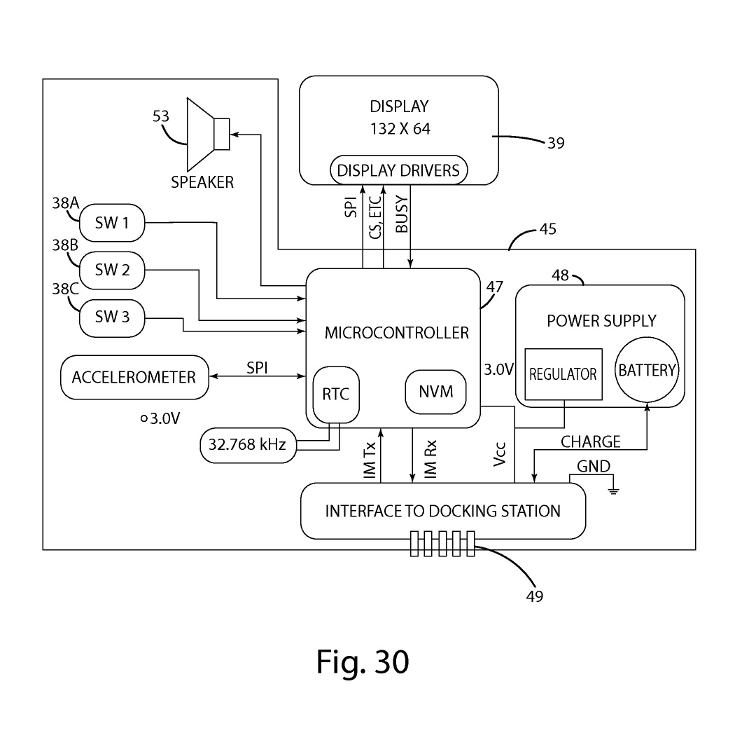

The components of several embodiments of different inhaler modules are described in connection with FIG. 30. The printed circuit board 45 can generally include a microcontroller 47 joined with a power supply 48. The microcontroller can control the user interface on the display 39, store health-related information in a memory collected during use or input by a user via buttons 38a-g, keep track of time, and manage any communications with the docking station to which the inhaler module is mounted. For example, the microcontroller can be used to generate questions relating to health on the display and/or related to the administration of a dose using the inhaler. A user can respond to those questions by inputting responses via the buttons 38a-c. Alternatively, the inhaler module may not include buttons 38a-c and instead a user can respond to the questions by inputting responses via the accelerometer. In another alternative embodiment, the inhaler module may not generate questions relating to health. If questions are posed, responses to those questions can be stored in the memory by the microcontroller and later transferred to the docking station. The microcontroller 47 also can store the health-related information to prevent reading of the information except through the docking station 70 (described below). The inhaler module microcontroller may be reprogrammable via the dock connection and may include a boot loader located in the microcontroller. This allows firmware updates even after the unit has been purchased.

In general, the power supply 48 can include a battery, a voltage regulator and an over/under voltage protection circuit. The battery can supply power to the device when it is not mounted on the docking station. The battery can also be rechargeable when it is mounted in the docking station. A lithium-ion or lithium polymer battery or other suitable small sized batteries can be used. In general, the battery life, with multiple rechargings, can be one, two, three or more years with normal usage. The battery can be incorporated into the circuit board 45 or other component of the inhaler module so that it does not rattle or come loose during the lifetime of the inhaler module. By including the battery in the inhaler module 30, rather than the mounting element 10, it can be easier to recharge the battery and/or provide access to information on the inhaler module even when the module is not attached to the inhaler via the mounting element 10.

The voltage regulator of the power supply 48 can adjust the voltage from the battery to a usable range for all of the components of the inhaler module 30. It can generally include a low operating current to reduce the drain on the battery and also have an input voltage range over the full range of battery value operating parameters. The power supply can also be configured to protect against lower voltage on the input, that is, when the battery is charging in a docking station, and also can cut off the battery when the battery voltage is too low to prevent excessive draining of the battery and avoid damage to the battery. Although the power supply 48 described herein can include the multiple components, namely, the battery, power regulator and circuit protection, other power supplies suitable for use with the inhaler module 30 can be used as desired.

The display 39 can be virtually any type of display screen, for example, a liquid crystal display, a cholesteric liquid crystal display, an OLED and/or similar type of graphical user interface, such as a Kent display available from Kent Displays of Kent, Ohio. As mentioned above, there is a variety of information that can be displayed on the screen 39. For example, the screen can display (a) the type of inhaler to which the module is joined, (b) a dose meter, (c) a theme image, (d) a use or compliance status, (e) a charge status; (f) a clock or timer; and/or an alert. For example, with regard to the inhaler type, an icon can be used to represent whether the inhaler is a maintenance inhaler or a rescue inhaler. With respect to the dose meter, the amount of medicine remaining in a respective inhaler, and more particularly, a canister, can be available as a percentage or as a number or other identifier. With regard to the theme image, multiple available images can be stored in the memory of the inhaler module and displayed on the display. With respect to the use status and alerts, the display can provide text which generally describes any of the following: whether the scheduled maintenance dosage has been taken, whether there is a scheduled maintenance dose, whether the scheduled maintenance dose has been missed, whether multiple scheduled maintenance doses have missed, whether too many doses have been taken, whether the battery is low, whether most or all of the doses have been administered, and/or whether the medication is expired. The display of the number of remaining doses or medication remaining can allow a patient to plan ahead for medication requirements, which can include carrying a replacement canister or visiting a pharmacy to refill the prescription for a replacement canister. Data related to the foregoing can be stored in memory on the printed circuit board coupled to the microcontroller, and/or on the docking station 70, and/or in microcontroller internal memory. As described below, the docking station can output signals related to the collected information so that others can be alerted to the status of compliance, battery usage, as well as other features or operating parameters.

As illustrated in FIG. 30, the inhaler module can also include a speaker 53 or other type of and transducer. The speaker can provide audible output such as an alert to a patient as to when a dose is due. The transducer can provide audio alerts in the form of a simple tone or series of tones via the speaker. The inhaler module can also include a real time clock (RTC) that tracks time and date. The microcontroller can accordingly record and store dosing events and input provided by the user via the buttons 38a-38c, if buttons are included on the inhaler module.

The inhaler module 30 also can include an accelerometer as shown in FIG. 30 which can be used to wake the device from sleep mode upon sufficient motion, such as shaking of the inhaler module 30. The accelerometer also can determine if the inhaler was sufficiently shaken to mix the medicine and the propellant in the canister 107 of the inhaler 100, which will be discussed in more detail below in connection with the third and fourth aspects. In one embodiment, the accelerometer may detect forces in three axis up to 8 g for shake detection. The accelerometer may communicate its output with the inhaler module microcontroller.

Referring to FIGS. 30, the inhaler module 30 can include data and power contacts 49 for recharging and communicating with the docking station. The particular contact can be located within a pocket to protect those contacts on the module or the docking station from unintentional shorting or draining. Of course, when the hardwired contacts are not desired between the inhaler module and the docking station, these elements can be replaced with optical, wireless, infrared, laser, inductive coils or other wireless features to transfer data from the inhaler module 30 to the docking station 70 and vice versa. Optionally, the data and power contacts 49 can be replaced with a suitable USB or micro USB as desired.

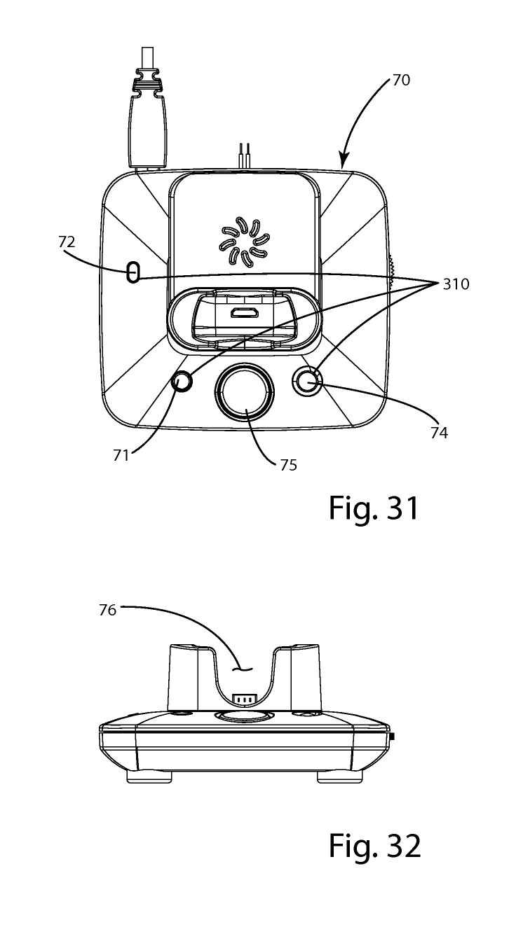

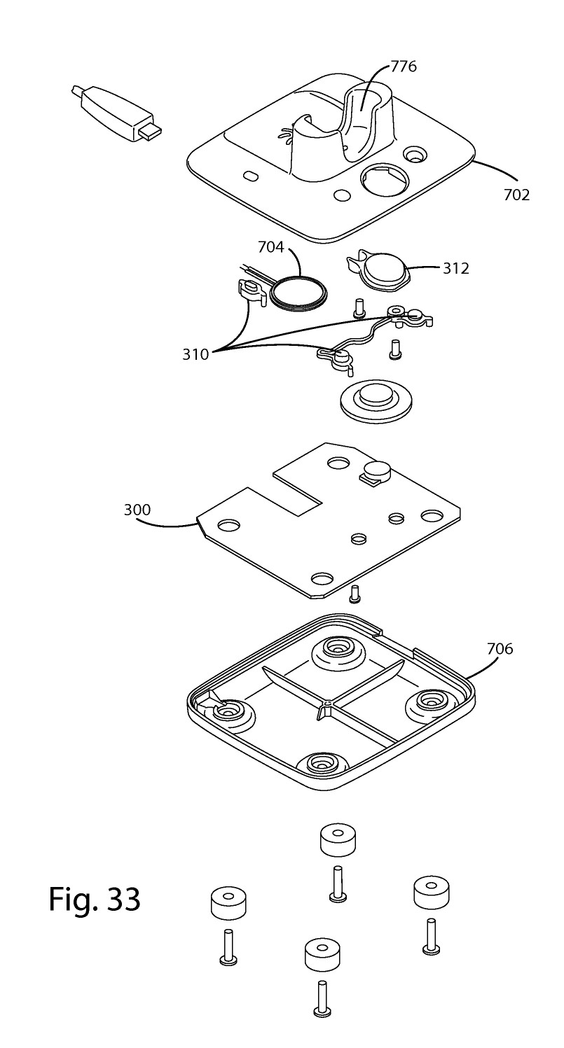

The inhaler module 30 can connect with the docking station 70 to exchange data and recharge the battery. The docking station 70 can handle sending the data forward and managing information and data that is to be loaded from the docking station from some other external network, database or computer to the inhaler module. Some examples of docking stations are illustrated in FIGS. 31-35. The primary differences between the two docking stations illustrated in FIGS. 31-34 is that the one in FIGS. 31-34 docking stations include one port 76 designed for a single inhaler module 30, whereas the other docking station, shown in FIG. 35 is designed for multiple inhaler modules. Such a multiple inhaler module docking station may be desirable where multiple inhaler modules are used on different inhalers within the household, for example, one on a rescue inhaler and another on a maintenance inhaler.

The docking station of FIGS. 31-33 will be described in detail here. The docking station 70 can include a top housing 702, a plurality of indicators 310, a printed circuit board 300, a speaker 704, a pushbutton 312, and a bottom housing 706. In the current embodiment, the top housing 702 and circuit board 703 define a port 76 that accepts an inhaler module. The top housing 702 and bottom housing 706 are joinable to create a docking station 70 housing that protects the docking station components.

In general, regardless of the type of docking station, the stations are adapted to receive inhaler usage data stored on the inhaler module and transferred to the docking station.

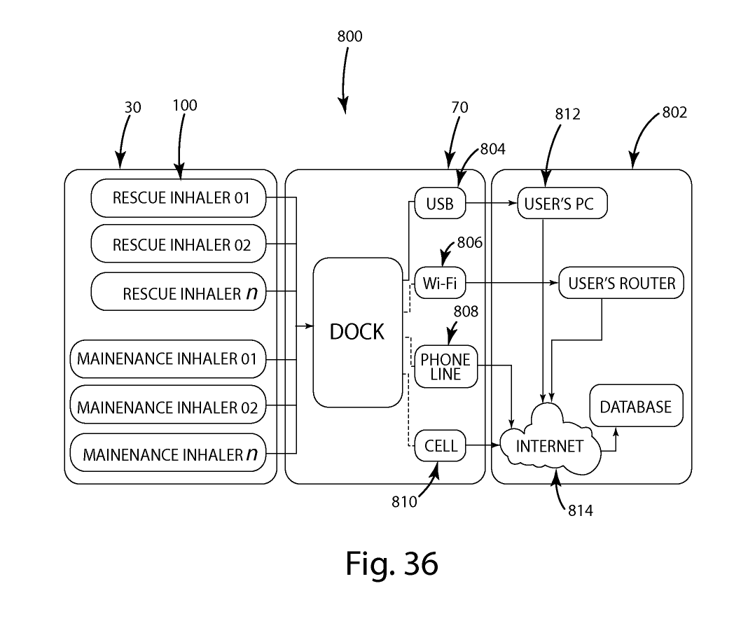

As shown in FIG. 36 and generally designated 800, the docking station 70 can communicate with a computer or network, generally designated 802, via USB 804, cellular 810, wireless 806, infrared, phone 808, modem, or any other conventional communication connection. For example, the docking station 70 can communicate with a computer 812 via USB 804. A computer program running on the computer can read data from the docking station 70 over the Universal Serial Bus 804 and communicate with an external server through the computer's internet 814 connection. The computer program can adjust various parameters on the dock and also update include a charger to charge the batteries of the inhaler module time and other settings. In some embodiments, these updates can be done utilizing a web based application in communication with the docking station and/or inhaler module. In embodiments where the docking station 70 includes a wireless or cellular connection, the data can be transmitted to server on the internet, without the use of a personal computer. The data may be encrypted and packaged according to any number of appropriate protocols and standards. In the current embodiment, data is encrypted using AES128, DSN, and TCP/IP. In some embodiments, the data may be sent to an intermediate server where the data resides until requested by the final server.

As mentioned above, the docking station 70 can include one or more ports 76 into which the respective inhaler modules 30 can be positioned. The inhaler modules can be positioned in the port whether or not they are connected to the inhalers 100. For example, the docking station can be configured so that it accommodates the physical structure of the inhaler module 30 while the inhaler module is attached via the mounting element 10 to the inhaler 100.

In general, the docking station 70 can perform one or more functions. First, it can send or receive data to the inhaler module 30 when the inhaler module is docked. The docking station 70 can also transfer inhaler module data to a server, network, or computer and likewise transfer data from the server, network or computer to the inhaler module. This may be performed in a variety of manners, for example, via USB, WiFi, modem, cellular or other connections with the network, server, or computer. The docking station also can include a charger that charges the battery of the inhaler module 30 when the inhaler module 30 is appropriately docked.

The docking station 70 can include a printed circuit board 300, a power charger 302 for the battery in the inhaler module, and a power supply connector to connect the docking station to a power supply and/or to a server. Depending on the connection with an external server, network or computer, the docking station can also be equipped with an appropriate phone modem, cellular antenna and/or WiFi components.

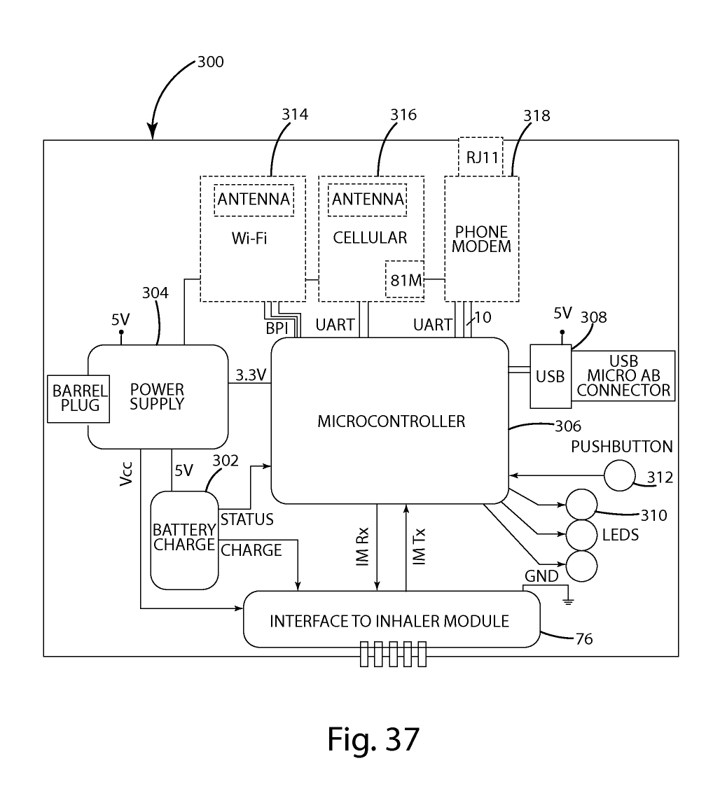

An exemplary circuit block diagram for a docking station is illustrated in FIG. 37. The docking station of the current embodiment includes a power supply 304, a battery charge circuit 302, a microcontroller 306, a USB circuit 308, a port or interface to the inhaler module 76, a plurality of LEDs 310, and a pushbutton 312. In alternative embodiments, the battery charge circuit 302 is located in the inhaler module instead of on the docking station. In some embodiments, there is no user input provided on the docking station and therefore the pushbutton 312 is not included on the docking station. The docking station optionally can include one or more of a WiFi circuit 314, a cellular circuit 316, and/or a modem 318.

In general, the power supply 48 regulates the voltage coming from an external power source for use throughout the docking station by the various circuit components. For example, the power supply can plug into mains power (100-120 VAC) and supply power to the module through a barrel plug type connector.

The battery charger 302 is responsible for charging the batteries of the inhaler module via the port 76 when the inhaler module is docked in the docking station. In the current embodiment, the battery charger 302 has independent control of charging and does not require microcontroller input. However, in alternative embodiments, the battery charger functionality and algorithm may be integrated into the microprocessor 306. In alternative embodiments, the battery charger 302 can be located in the inhaler module or located in an external charging unit, such as a cell phone charger suitable for use with the inhaler module.

The docking station microcontroller 306 can control a variety of different functions of the docking station including communication with the inhaler module, communication with the network, via one of the communication circuits, and the user interface, including the user input 312 (if included) and the docking station output 310. In the current embodiment, the docking station microcontroller 306 is can simultaneously handle USB communications along with communication to the inhaler module. The docking station microcontroller 306 can perform AES128 encryption and decryption. The microcontroller can also run a TCP/IP stack, for example in embodiments that employ WiFi. The microcontroller can be reprogrammed internally to allow upgrades.

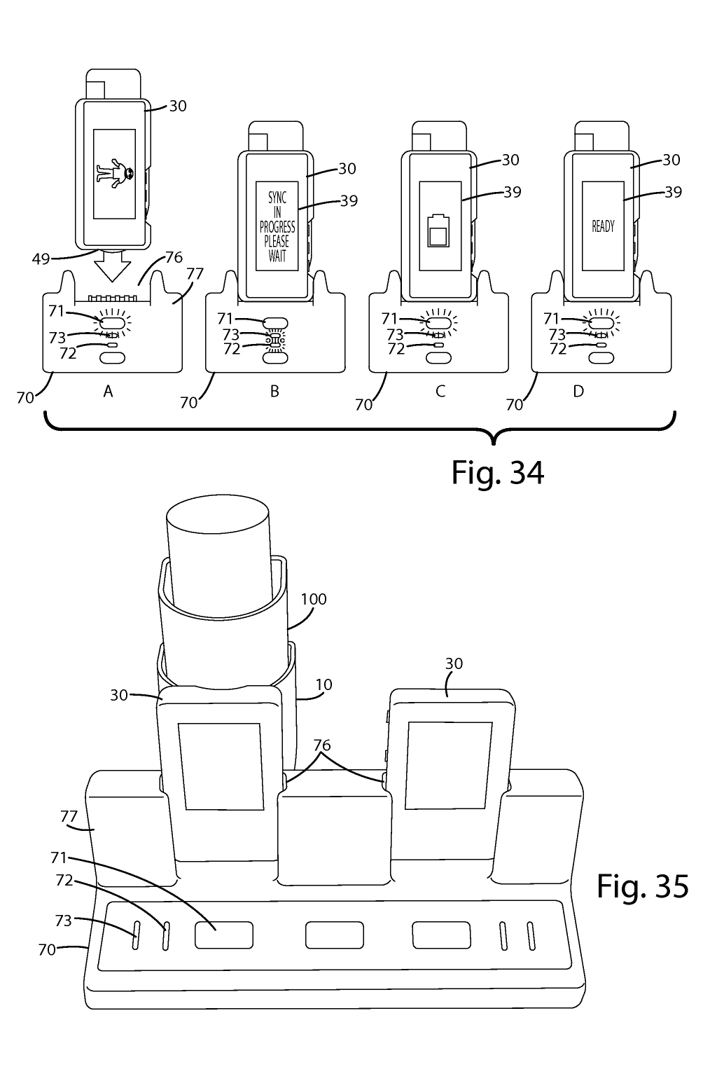

The docking station can be outfitted with a user interface that includes one or more indicators 310. As illustrated in FIG. 31-33, the indicators can include a refill reminder indicator 74, a data transfer indicator 72, and a compliance indicator or alert indicator 71. These indicators can be in the form of a light or other visual indicator that is actuated to indicate certain events. An example of a suitable indicator is one ore more LEDs or other lights, capable of flashing and/or illuminating in different solid colors. In alternative embodiments, such as the docking stations illustrated in FIGS. 34-35, the refill reminder indicator may be replaced with a battery indicator 73. In other embodiments, a docking station may include different indicators.

The battery indicator 73 can show the charging status of the battery in the inhaler module 30 by the docking station 70. As an example, the indicator 73 can be off when there is no inhaler module docking station. When the inhaler module is in the docking station is and charging, the battery status indicator 73 can be flashing. When the battery of the inhaler module 30 is fully charged, the indicator can be of a solid color. If the battery of the inhaler module has failed, the indicator 73 can rapidly flash.

The data transfer indicator 72 can indicate data transfer, and particularly the data transfer status between the inhaler module 30 and the docking station 70 when the inhaler module is positioned in the port 76 and in communication with the docking station 70. For example, the data transfer indicator 72 can be off when there is no inhaler module docked. When the inhaler module 30 is in the docking station and transferring information, the data transfer indicator 72 can be flashing light of a first color, for example, red. When the process of data transfer is complete, the data transfer indicator 72 can be a solid second color, for example, green, for 30 seconds and then turn off after that period. Alternatively, when a fatal error in data transfer has occurred, the indicator 72 can flash yet another color, for example, red, to indicate the same. This same indicator 72 can also indicate the status of data transfer to a network, computer or server in a similar fashion.

The docking station 70 can also be configured with a compliance indicator 71. This compliance indicator can be helpful in that it can show an overview of user compliance upon a brief inspection. For example, the compliance indicator 71 can be helpful to a caregiver, such as a parent, and can provide an easy way to confirm that their child is complying with a medication regimen simply by reviewing the indicator and its status. In some embodiments, the inhaler module displays the current alert if the user pushes a button 75 to wake it up. The compliance indicator can sometimes be referred to as a compliance-at-a-glance indicator. The compliance indication can have a check mark icon or some other indicia to make it clear that it is the compliance light.

In general, the compliance indicator 71 can be in communication with the microcontroller 47 of the docking station 70 and/or the inhaler module 30. The microcontroller 47 can process the data downloaded from the inhaler module, for example, the compliance data regarding the patient's use of the inhaler 100 at prescribed times and/or at prescribed frequencies and/or numbers of times of use since the last download. The microcontroller can process that information, compare it to pre-selected values for compliance data, and output signals to the compliance indicator 71. The compliance indicator can subsequently indicate, visually or audibly, the general compliance level of the patient using the inhaler to which the inhaler module 30 is attached.

The following is an example of how the microcontroller might process downloaded compliance data. To begin, the inhaler may be used by a user over a two week interval between dockings on the docking station. During that time, the user's asthma medication compliance regimen may specify maintenance inhaler use three times a day, for a total of forty two total doses administered over the two weeks. The user, however, may only use the inhaler thirty times over the two weeks. That compliance data, related to the thirty uses may be recorded on the inhaler module 30, and downloaded to the docking station. The docking station microcontroller may process as the compliance data. In doing so, it may compare the actual doses, thirty, to the prescribed doses, forty two.

In comparing the thirty actual doses to the forty two doses, the microcontroller may output a value of 72% compliance (actual doses divided by prescribed doses). This compliance level, 72%, may then be compared to a pre-selected compliance level of, for example, 69%. Because the measured compliance value of 72% is greater than the pre-selected compliance level, the microcontroller may send a signal to the compliance indicator 71 that causes the compliance indicator 71 to indicate the compliance level is satisfactory. For example, the compliance indicator may output a green light. If, on the other hand, the compliance indicator had been less than 69%, the microcontroller may have sent a signal to the compliance indicator 71 that causes the compliance indicator to indicate the compliance level is unsatisfactory. For example, the compliance indicator may output a red light. To the user and a caregiver of the user, such output, that is a red or green light, can generally signify the compliance level of the user relative to a prescribed medication compliance regimen. The docking station may be placed in a location visible to the caregiver so that they can readily ascertain the compliance level of the user.

Of course, the microcontroller can perform other operations on the compliance data to output an indication from the docking station indicative of compliance levels. Incidentally, compliance level can refer to relative degrees of compliance, like percentages of compliance, or can simply refer to compliance or no compliance. In the present example, the microcontroller may compare the thirty actual doses to some other pre-selected number that indicates a compliance level. For example, a value of thirty five uses may be selected as “satisfactory”. Because the value of actual doses, thirty, is less than the satisfactory thirty five doses, the microcontroller may control the compliance indicator 71 to output a signal, such as illumination of a red light or emission of an audible tone, which indicates an “unsatisfactory” compliance level.

The following is an example in which the compliance indicator 71 includes a set of lights, for example, red, green and yellow lights (optimally LEDs) that can be independently illuminated based on compliance data transferred from the inhaler module 30 to the docking station 70. In this example, the compliance indicator 71 can illuminate the green light to indicate that a user is compliant in compliance. When the user has missed the last dose, the compliance indicator 71 can illuminate the yellow light to indicate that there has been recent non-compliance. When the user has missed multiple doses, the compliance indicator 71 can illuminate the red light to indicate that that there has been multiple events of non-compliance. In order to assist the inhaler user with remembering to take the medication, the inhaler module can be programmed to alarm the user when it is time to take a dose. When the user misses an alarm, but is still within a predefined window of time after the alarm, the compliance indicator 71 can flash the light to indicate that the user has not taken the medication yet. In this embodiment, the flashing can maintain the color of the compliance indicator so as to impart both the level of compliance and whether a window for taking medication is open.

In another example with a similar set of lights, the compliance indicator 71 can illuminate the green light to indicate that a user is compliant at least a certain percentage of the time, for example, 95% of the time in the recent past. While the recent past can be any amount of time, it optionally can be the amount of time, as measured by the inhaler module internal clock or clock in the docking station, since the last download of information from the inhaler module 30 to the docking station 70. The compliance indicator 71 can illuminate the yellow light, which indicates that the user has been compliant for another percentage of the recent past, for example, 70% to 94%. The compliance indicator can illuminate the red light or a red flashing sequence to indicate to a caregiver that the user's compliance just has dropped below a certain percentage, for example, 69%, or that the user has overused an inhaler. Upon learning this information, based on compliance level signals output through the compliance indicator 71, the caregiver can then take appropriate action with regard to the user and/or the treatment regimen.

The compliance indicator 71 can flash the yellow light to indicate to the user and a caregiver that the inhaler is running low on medicine. The caregiver and/or user can then take appropriate action and refill the inhaler or ensure that the user has an extra canister for the inhaler on hand. The compliance indicator 71 can illuminate the red light to show that the inhaler to which the inhaler module is attached has expired. A caregiver or user can subsequently replace the expired inhaler with a new inhaler. The indicator can maintain its current status until the docking station 70 is synchronized with the inhaler module 30 and the status information transferred from the inhaler module to the docking station changes.

Optionally, the level of compliance can be indicated by the compliance indicator 71 in a different way. For example, the indicator can include a red light and a green light. The green light is can be illuminated when the compliance data indicates the inhaler was property properly used at the prescribed times at least 80% of the time in recent past. The red light is can be illuminated if that the compliance data indicates the inhaler was used at the prescribed times less than 80% of the time in the recent past. As another example, the compliance indicator 71 can be a speaker. The speaker can emit an audible tone when compliance is less than 75%, and no tone when the compliance is grater than 70%. A variety of other indication output signals can be generated to indicate compliance levels depending on the application.

FIG. 34 illustrates some examples of the various indicators indicating different operating parameters. For example, in FIG. 34 part A, the inhaler module 30 is initially plugged into the port 76 of the docking station 70. There, the indicator 71 indicates from the previous docking of the inhaler module 30 that the dosing was in compliance with a treatment regimen. Therefore, the indicator 71 is illuminated.

In part B of FIG. 34, the inhaler module 30 is synching with the docking station, and the data transfer indicator 72 is indicating the download of data from the inhaler module to the docking station 70. During this time, the docking may also begin to charge the battery of the inhaler module 30, in which case the charging indicator 73 illuminates. In part C of FIG. 34, the docking station 70 continues to charge the battery of the inhaler module 30. At this point, the compliance data downloaded from the inhaler module has been transferred to the docking station 70. The downloaded compliance data represents a sufficient compliance level with the treatment program. Therefore, the compliance indicator 71 illuminates to indicate a positive compliance history, for example, the indicator can be a green illuminated light. If the compliance data represents insufficient compliance levels, then the compliance indicator 71 can be a red illuminated light, again, indicating poor compliance. Of course, a caregiver reviewing the docking station as illustrated in this part C of FIG. 34 could take appropriate action to enhance compliance levels as appropriate. In part D of FIG. 34, the compliance indicator remains illuminated to indicate the status of compliance. The inhaler module display 39 can indicate that the inhaler module is ready to be removed from the docking station 70 whenever desired, and that the battery is fully charged.

Optionally, the indicator 71 can be an LED that maintains a color from the last data upload from the module to the docking station. The LED can change color (if compliance changes) when the next data upload occurs. The LED can turn off if the dock is unplugged or looses power. The LED can stay off until the next data upload.

After the data from the inhaler module 30 is downloaded to the docking station, the docking station can store the data in memory until it is subsequently transferred to another computer, a network or a server where that data can be shared with patients, parents, nurses, physicians, medical researchers, pharmaceutical companies of course, after being appropriately encrypted or otherwise anonymized and/or encrypted to protect patient information. The data can then be used to monitor the treatment regimen over a given time period. In some embodiments, for example because of potential privacy concerns, no data is stored on the docking station. The LED color can be maintained for compliance/expiration until the device is docked a second time resetting the LED setting. In other embodiments, the status of the LED can be changed if a docked inhaler sits longer than a threshold amount of time in order to indicate that a user has missed one or more doses.

In general, the docking station can collect, store and transfer a variety of data, from the inhaler module to a network, database or other computer. Such data can include a patient's: 1) identification; 2) compliance with a treatment regimen; 3) the prescribed medication; 4) actual dosage taken during each use of the inhaler; 5) responses to questions posed via the inhaler module; and 6) status of the inhaler medication, and other related compliance data.

### DESCRIPTION OF EMBODIMENTS OF THE THIRD ASPECT

The third aspect relates generally to tracking and indicating the mixing status of the contents of the medication canister of an inhaler based on accelerometer output. For example, the mixing status of the contents of the medication canister can be tracked with an accelerometer and the status can be presented to the user with a display or another indicator.

As with the other embodiments, the mounting element of this embodiment can be suitable to mount a variety of inhaler modules to an inhaler. The inhaler module can be configured to record usage information of the inhaler and to provide direct feedback to the user in certain circumstances. As described above, the inhaler module can include a screen through which information is conveyed to a user. Different types of information can include information be displayed via the sensor, including dosage, power source information, and other patient-related data. For example, the inhaler module can provide reminder alerts to take medication with an audible tone or otherwise display an alert on the display. The inhaler module can also show compliance history and provide congratulatory indications after doses are taken within the desired time frame of a medication regimen.

In one embodiment of the third aspect, the inhaler module can record the time of a “shake” event in memory. The accelerometer can be programmed to provide an output when a shake event occurs according to predefined characteristic. Alternatively, the accelerometer can provide, from time to time, accelerometer output and the microprocessor can process the accelerometer output to determine when a shake event occurs. For example, the microcontroller may be programmed to recognize a shake event if the accelerometer output indicates that a pre-defined threshold of force is surpassed in all three axes. A shake event can be defined differently depending on a number of factors, for example different medications may need a different amount of shaking at a different level in order to register as a shake event. Generally, a shake event can be defined by a sequence of vibrations that exceed an acceleration threshold for a predetermined amount of time.

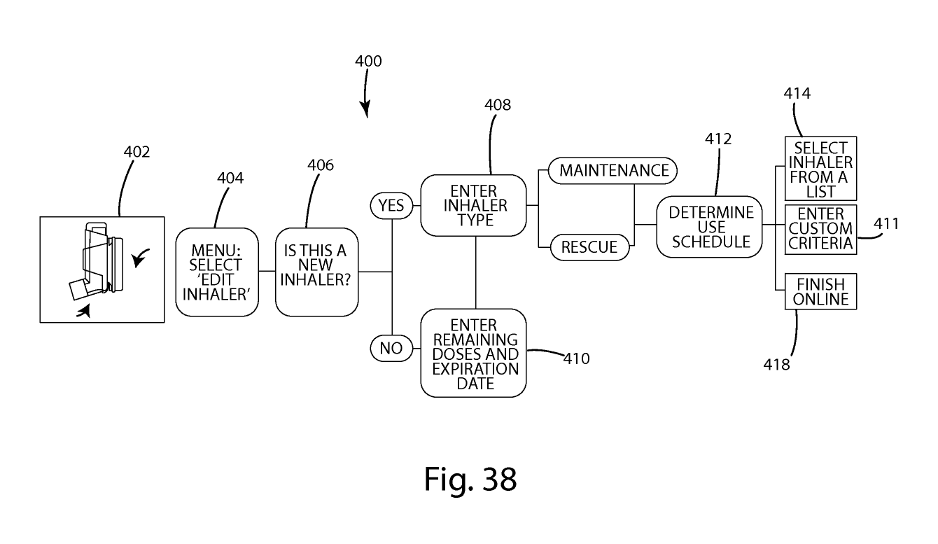

Pairing the inhaler module with an inhaler is the procedure that allows an inhaler module to be configured to work with different inhalers or different canisters of medication for any given inhaler. In some embodiments, where the inhaler module is associated permanently with an inhaler and particular type of medication, the pairing process may be unnecessary. An exemplary method of pairing is illustrated in the flow chart of FIG. 38, and generally designated 400. The method of pairing an inhaler module with an inhaler includes: attaching the inhaler module to the inhaler 402, selecting the pairing option from a menu on the inhaler module or a computer software program that interfaces with the inhaler module, indicating whether the inhaler is new or used 406, if the inhaler is used, entering the remaining doses and expiration date 410, if the inhaler is new, entering the inhaler type 408 (i.e. maintenance or rescue), determining the use schedule 412, selecting the inhaler from a list 414, entering any custom criteria 411, and if applicable, finish the pairing process online 418 by interfacing with the compliance reporting subsystem. By selecting the inhaler from a predetermined list, the inhaler module can be populated with preset maintenance schedules, maximum dose limits, and an expiration date. The patient or healthcare professional may be able to override these preset configurations with different prescription schedules, dose limits, and expatriation dates. In some embodiments, this information can be changed over the network, when the inhaler module is docked in the docking station.

In some embodiments, the inhaler module graphical user information can support user customization. For example, various themes may be available on the inhaler module, where each theme includes multiple status, ready, alert, and reminder screens.

One embodiment of a method for indicating mixing status of a medication canister for an inhaler includes the steps of sensing that the inhaler module has been undocked; indicating an instruction to shake the inhaler in order to mix the contents of the medication canister; shaking the inhaler shakes to simultaneously shake the medication canister and the accelerometer; generating accelerometer output that can be used to track the mixing status of the medication canister; and indicating the mixing status.

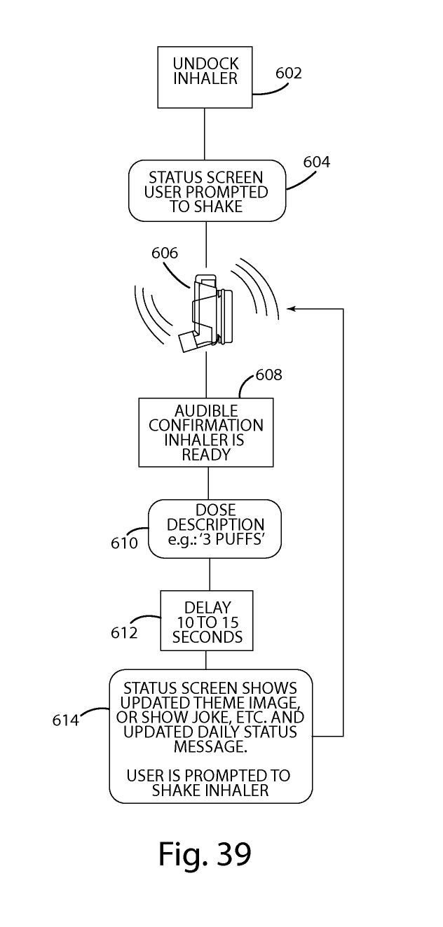

Referring to FIG. 39, one embodiment of a method for indicating mixing status of a medication canister for an inhaler is illustrated, and generally designated 600. The method includes the steps of: undocking the inhaler 602; instructing the user to shake the inhaler 604; generating accelerometer output in response to the user shaking the inhaler 606; optionally, displaying an animation on the screen in response to the accelerometer output. Continuing with the method, indicating, via an audio, visual, or other type of indication, that the medication canister is sufficiently blended 608; prompting the user to take a puff and optionally displaying other dose information, such as the number of doses remaining 610; initiate a countdown timer to delay 612, for example in the depicted embodiment a 10 to 15 second delay is initiated; in response to the countdown timer completing providing a status screen and if additional puffs are to be performed prompting the user to shake the inhaler 614; if additional puffs are to be performed steps 606-614 can be repeated for however many puffs are to be taken. After the dose is complete, the inhaler can be placed back on the dock and that information can be uploaded to the dock and communicated to a web server or other compliance subsystem.

As with any of the embodiments, this embodiment of the apparatus can be utilized for indicating mixing status of an inhaler medication canister. The apparatus can generally include an inhaler module, an accelerometer, a mounting element, an indicator, and a microprocessor. For example, the inhaler module can collect information related to use of the inhaler. The accelerometer can generate accelerometer output signals in response to shaking. The mounting element can removably mount the inhaler module in a spatial location on the inhaler, wherein, when mounted, shaking the apparatus simultaneously shakes the medication within the medication canister and shakes the accelerometer. The indicator, for example a speaker, LED, or display, can communicate information to a user of the apparatus. The microprocessor can be in communication with the accelerometer, for example by electrical connection, and the microprocessor can be programmed to 1) determine if the inhaler was sufficiently shaken to mix the contents in the medication canister, such as the medicine and the propellant, based on the accelerometer output signals and 2) control the indicator to indicate the mixing status to the user of the apparatus.

In one embodiment, the inhaler module display can display an animation on the screen. For example, the animation may move across the screen and the speed of the animation can vary as a function of the shaking such that the animation completes at the time in which the medication canister mixing is complete.

### DESCRIPTION OF EMBODIMENTS OF THE FOURTH ASPECT

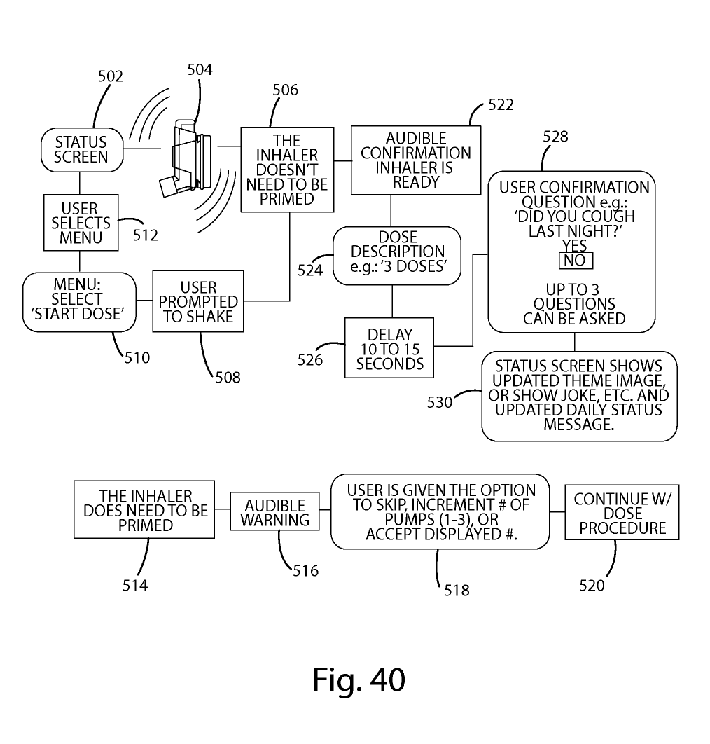

The fourth aspect relates generally to reminding a user to prime the inhaler. One embodiment of a method of utilizing the inhaler and inhaler module with the priming reminder is illustrated in FIG. 40, and generally designated 500.

In the illustrated embodiment, the user begins the inhaler use method from the status screen 502. The user either 1) selects a menu 512 and a start dose option 510 from the menu, where the user is prompted to shake the inhaler, or 2) the user shakes the inhaler 504. In alternative embodiments, such as using the primer reminder feature with the inhaler module depicted in FIGS. 1-5, which does not have any tactile user input, the user shakes the inhaler.

Upon sufficient shaking, the inhaler module may record the timestamp of the shake event and determines whether the inhaler needs to be primed. In the current embodiment, this determination is made by comparing the timestamp of the current shake event with the last timestamp indicating that the inhaler was utilized. If the inhaler does need to be primed 514, then an audible, visual, and/or other signal 516 can be provided to the user. The user can be given the option to skip the priming, change the number of priming pumps, or accept the number of priming pumps performed 518. In alternative embodiments, this step may not allow the user to skip the priming step.

Once the priming procedure is complete, the dosing procedure can continue 520. Before the dosing procedure begins, the inhaler module may present a health related inquiry that requires user input. An audible or other confirmation signal is provided to indicate that the inhaler is ready 522. A dose description or dose instruction is provided to the user 524. A delay can be provided before requesting input from the user 526. The inhaler module then asks one or more user confirmation question or a health related inquiry 528. For example, the user may be asked to confirm the inhaler dose was taken, whether the user coughed last night, or to rate their current breathing capacity. After all questions posed by the inhaler module are answered, the inhaler module displays an updated status screen 530, which can include showing a decreased number of available doses. As a motivation to follow through with the confirmation, an acknowledgment or reward might be shown. In addition to confirming inhaler use, answers to the question(s) posed on the inhaler module can offer additional data points for evaluation. Further, the time differential between the shake time stamp and the answer time stamp is an additional data point for evaluation.

In another embodiment, the timestamped data can help facilitate proper priming of the inhaler. By recording timestamps about when and how often the inhaler is utilized, it is possible for the inhaler module to indicate whether the inhaler needs to be primed before dosing. For some asthma medication, it is recommended that the inhaler be primed if it has not been used for a prolonged period of time. Accordingly, in response to the inhaler being shaken and woken up, the controller in the inhaler module can be programmed to compare the last recorded timestamp of dosing to a time threshold for priming. The time threshold can vary depending on a number of different factors. In the current embodiment, the threshold varies with the type of medication being used in the inhaler, and is set as a function of the type of medication at the time the inhaler module is paired with the inhaler. Accordingly, the timestamped data assists the user in identifying when it is appropriate to prime the inhaler. This can be particularly difficult because sometimes a user will not be able to remember when the inhaler was used last. Because some users have a difficult time remembering when to prime their inhaler, some users make it a habit to prime their inhaler before ever use, which can result in wasted doses of medication. The timestamped data of this priming reminder feature allows users to avoid both under-priming the inhaler and over-priming the inhaler.

It should be understood that timestamped data refers to any data associated with a date, time, or any other indication of when one piece of data was obtained relative to when another piece of data was obtained. The timestamps can be absolute or relative.

### DESCRIPTION OF THE EMBODIMENTS OF THE ADDITIONAL ASPECTS

Another aspect includes an inhaler compliance system that includes an inhaler module, docking station, and compliance reporting subsystem. As with the other embodiments, the inhaler module can attach to an inhaler without the use of tools via an elastomeric holder. Again, just as with the other embodiments, the inhaler module can provide status information such as remaining doses and when the next dose is due, reminder alerts, compliance history, and can reinforce enthusiastic compliance via congratulatory visuals after each dose taken within the desired timeframe. In addition, the inhaler module can communicate with the compliance reporting subsystem via the docking station.

The docking station can transfer data to a network, such as the Internet, so that it can be appropriately shared with one or more of: patients, parents, nurses, physicians, caregivers, medical researchers, and/or pharmaceutical companies. For example, adult patients can monitor their own compliance record for a given time period. Parents of younger patients, who typically may rely on their child's assurance that the medication was taken when the child is at school or day care, for example, can view the database to check on their children's compliance to determine the frequency of administration.

School nurses can use the system to obtain patient compliance data for their student patients, which may be useful for younger patients who may forget to use their inhalers. Availability of the system at a school also can be helpful to student patients whose parents do not have the necessary computer or other equipment to download the information at home. For example, a school nurse can print a compliance report using the school's equipment and can either send the report home with the patient or mail or email the report to the patient's parents. Further, nurses at children's camps or the like, where children are away from home for an extended period of time, can use the device to ensure that the young patients are properly taking their medication. As discussed above, the nurses can then send a report to the patients' parents, who may wish to monitor their children when they are away from home.

Physicians or healthcare providers can use the database to monitor their patients' use of the device and to determine the effectiveness of the currently prescribed medication and dosage for each respective patient. A physician can then alter the patient's prescription based on the data. For example, a physician may determine, based on the information collected and transferred from the inhaler, that a patient's response to a certain medication is unfavorable. The physician can then increase or decrease the patient's dosage amount, or even change the type of medication prescribed. Additionally, the database also can allow physicians to identify which patients have problems with medication compliance. In response to this information, the physician can monitor these patients more frequently and/or prescribe an inhaler that includes an alarm or signal to remind the patient to use the inhaler at the prescribed time.

Optionally, the system may allow for the real-time transfer of data from a physician, for example, back to an inhaler. In one embodiment, a physician can use the database to enter a new dosage amount for a particular patient. The dosage change can be received by the computer of the patient's inhaler, which in turn automatically adjusts the amount of each dose that is dispensed each time the inhaler is actuated. Thus, the dosage can be changed immediately, without having to wait for the patient's next office visit.

The data collected by the inhaler module also can be of interest to medical researchers. The database can include patient identification information such as patients' geographic location, age, ethnic background, gender, height, weight or other biometric. Accordingly, researchers can use the database to identify trends in the patients being treated. For example, based on the data, it may be possible to determine that participants in a certain geographic region have experienced an increase in asthma or allergy medication administration. Such data may call for an investigation in the particular region to determine whether an environmental factor may be causing the increase in afflicted patients. Further, researchers can be encouraged to post any significant findings on the database. Thus, in addition to providing a source of data for researchers to analyze, the database can provide access to the latest research reports, keeping both patients and physicians apprised of the latest asthma and allergy-related news.

The data collected by the inhaler module also can promote clinical research performed by physicians or medical associations. As noted above, the database can identify each patient by various factors including age, location and ethnic background. The physicians can use the database to identify potential patients for clinical studies and trials. The physicians or others, such as pharmaceutical manufacturers, also can advertise on the database for willing participants. Additionally, the database can provide an easy way for the physicians to view and analyze patient data as part of the study. Because the data is obtained directly from the inhaler, the physician is assured that the data is relatively accurate, as opposed to relying on input from the patient.

Pharmaceutical companies may also have interest in the data to determine which medications, produced by both their own company and by competitors, are most effective and whether certain medications are more effective for particular patients. The company could then increase advertising for these particular medications and could share this information with physicians to promote the prescription of the medications.

In one embodiment, the database can be made available as part of a subscription service, to which individual patients, nurses, physicians, medical associations and pharmaceutical companies may subscribe, such as on an annual basis. To restrict access, subscribers can be required to enter a user name and/or password to gain access to the database. However, any suitable security measure may be used.

Optionally, in addition to accessing the data from the database, the subscribers may be permitted to contribute data to the database. For example, physician subscribers may upload or otherwise input data into the database regarding their own patients, clinical trials or other related information. The ability for subscribers to contribute data to the system may result in more widespread data collection, which may further assist physicians and researchers in identifying trends in patient data.

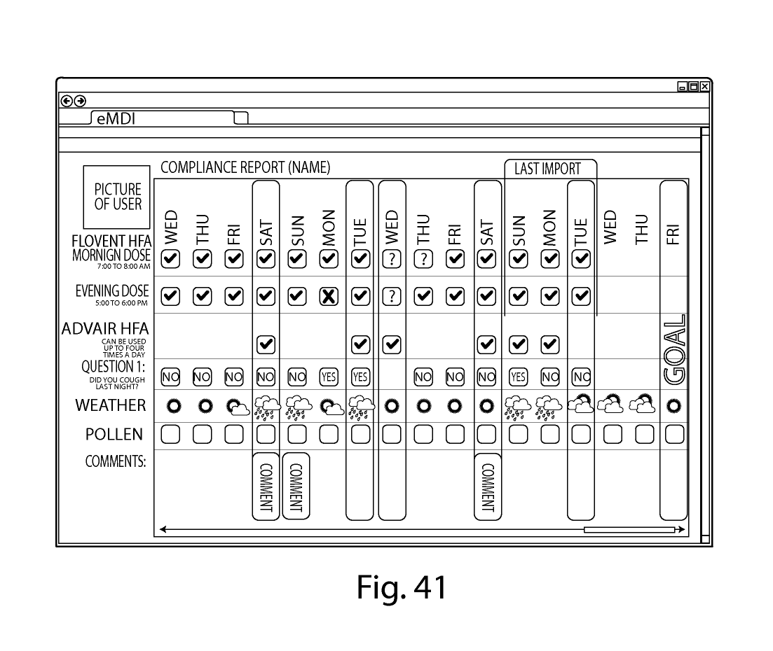

The data in the database may be presented in a compliance report. An illustration of an exemplary compliance report is shown in FIG. 41. In the current embodiment, information is presented to the user in layered and graphical manner. The compliance report allows users to identify trends visually. The compliance report is presented in a row and column format. Each column represents a different day and each row represents a piece of data. In the current embodiment, the data includes whether the patient took a morning dose, whether the patient took an evening dose, whether the patient took an Advair HFA dose, whether the patient coughed the previous night, the weather and pollen conditions, and any comments provided by the patient.

The compliance report can include a variety of other information. For example, in addition to general whether condition, the compliance report could include more specific weather information such as humidity, temperature and wind conditions. The compliance report could also include school scheduling information and extracurricular activities. Almost any timestamped data can be provided in the compliance report. In addition to timestamped data from the past, future timestamped data may also be presented with the compliance report. For example, the weather and school schedules can be presented as leading information ahead of the current date. This may enable health care professionals to take extra precautions based on previous trends, for example altering the dosages.

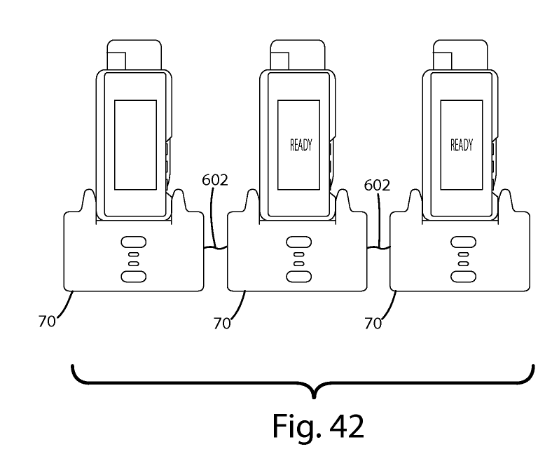

In another aspect, as illustrated in FIG. 42, multiple docking stations can be coupled together or coupled with a gateway device, either wired or wirelessly. Each docking station can accept an inhaler module or other healthcare related module and download data from the docked inhaler module. Each docking station 70 can communicate its data with the other docking stations. One docking station, which can be referred to as the main docking station, or a gateway can collect the data from the other docking stations and transfer the data to a database located on a network to be used in a compliance reporting subsystem. In alternative embodiments, each docking station may individually communicate with the compliance reporting subsystem. The connection between the docking stations may be a daisy chain or hub configuration and may be made with essentially any protocol capable of transferring data between the stations. In the current embodiment, a USB daisy chain configuration is illustrated where both power and information can be shared between the docking stations via USB cables 602. In alternative embodiments, the docking stations may be individually connected to a power source and communicate wirelessly using Bluetooth, WiFi, cellular, modem, or another wireless communication protocol and the associated circuitry.

In another aspect, the inhaler module 30 also can include one or more sensors or switches that can be used to sense activation of the inhaler or input from a user. For example, the sensors can be a light sensitive photosensor that is positioned adjacent a moving part of the inhaler or canister to sense when the inhaler is activated to administer a dose. Optionally, this light sensitive photosensor can be substituted with an infrared sensor, a laser sensor, a mechanical switch or some other element that can sense actuation of the inhaler. Further optionally, the inhaler module does not include any sensors that sense activation of the inhaler. The inhaler module accepts input from a user via the three switches located, and can instead rely on the user to input information confirming dosing via the buttons on the inhaler module 38a, 38b, 38c. In this embodiment, the inhaler module is free from sensors and does not have any electrical or mechanical communication with the inhaler. The inhaler module does not interfere with the usage of the inhaler, but still can provide instructions to the patient, query the patient, and accept input from the patient about the use of the inhaler. In this embodiment, the inhaler module does not include any sensors that can malfunction, break down, or interfere with the user's use of the inhaler, but the ability to record and track inhaler use can still be provided. Further, the timestamps collected from user input can provide additional data points that are not available in conventional inhaler systems that only track actual actuation of the inhaler via mechanical or electrical sensors. For example, there may be a timestamped inhaler module event when the user shakes the inhaler, when the user answers a health related inquiry before dosing the inhaler, when the user answers a confirmation question confirming that the inhaler was primed and the number of times the inhaler was primed, when the user answers a confirmation question confirming the number of doses that were taken, and/or when the user answers a health related inquiry after dosing the inhaler. Accordingly, multiple timestamps of inhaler module events may be associated with a single dosing event. Health care providers or the data compliance subsystem can analyze the timestamps and their relationship to identify trends and outliers. Recording and reporting this data can facilitate tracking of various metrics, including the time between waking up the inhaler module by shaking it and confirming that a dosage was taken. This information may be helpful in identifying improper use of the inhaler. For example, a child that is not actually using the inhaler might try to make it look like they are properly using the inhaler by shaking the inhaler, actuating the inhaler into the air, and then indicating that the inhaler was dosed. Put another way, using multiple timestamps it may be possible to identify if the user is too quick to register a confirmation after a dose maintenance session. The time between shaking the inhaler and indicating that the inhaler was dosed in this scenario can be very different from the time between shaking the inhaler and an actual accurate indication that the inhaler was dosed.

The above description is that of the current embodiment of the invention. Various alterations and changes can be made without departing from the spirit and broader aspects of the invention as defined in the appended claims, which are to be interpreted in accordance with the principles of patent law including the doctrine of equivalents. Any reference to claim elements in the singular, for example, using the articles “a,” “an,” “the” or “said,” is not to be construed as limiting the element to the singular.

The embodiments of the invention in which an exclusive property or privilege is claimed are as follows:

1. An apparatus for monitoring compliance with medication treatment regimen comprising:
an inhaler module that collects information related to the use of an inhaler, the inhaler including a medication canister,
an attachment element joined with the inhaler module, the attachment element including an attachment projection defining a recess between an attachment element base and a flange member,
a mounting element that removably mounts the inhaler module in a spatial location on the inhaler, wherein the mounting element includes an elastic body having
a first mounting portion that 1) defines an internal chamber adapted to receive the first inhaler portion and 2) exerts a gripping force on the first inhaler portion, and
a second mounting portion adapted to join the mounting element with the attachment element,
wherein the first mounting portion of the elastic body is stretchable from a relaxed mode to a stretched mode so that the inhaler can be installed in the elastic body in the stretched mode,
wherein the second mounting portion of the elastic body defines a hole having a periphery, wherein the flange member of the attachment element is adapted to fit through the hole when the periphery is stretched and is adapted to return to a relaxed mode so that it surrounds the attachment element base.

2. The apparatus of claim 1 wherein the attachment element base is integrally formed with a rear housing portion of the inhaler module.

3. The apparatus of claim 1 wherein the elastic body of the mounting element defines a recess that is configured along an edge of the elastic body, wherein the recess is dimensioned and sized so that the second inhaler portion or the mouthpiece of the inhaler fits at least partially within the recess.

4. The apparatus of claim 1 wherein the first mounting portion includes one or more apertures to increase flexibility of the elastic body.

5. The apparatus of claim 1 wherein the periphery of the hole engages the attachment element base and prevents rotation of the attachment element base within the hole.

6. The apparatus of claim 1 wherein the flange member is larger than the periphery of the hole in the second portion of the mounting element so that the flange member effectively captures a portion of the elastic body between the flange member and the attachment element base.

7. A method of indicating a compliance level related to a medication treatment regimen comprising:
providing an inhaler module that mounts piggyback to an inhaler with a mounting element;
storing asthma medication treatment compliance data related to the dispensing of medication from the inhaler with the inhaler module;
downloading the asthma medication treatment compliance data from the inhaler module to a docking station;
comparing the asthma medication treatment compliance data to pre-selected values for the asthma medication treatment compliance data;
outputting a first signal via an indicator joined with the dock, wherein the signal is indicative of the level of asthma medication treatment compliance by a user of the inhaler.

8. The method of claim 7 including locating the docking station in a location visible to a caregiver so that the caregiver can readily ascertain the compliance level of the user of the inhaler.

9. The method of claim 7 wherein the method includes docking the inhaler and said signal is indicative of the level of asthma medication treatment compliance by the user of the inhaler since the last time the inhaler was docked.

10. The method of claim 7 wherein the signal is indicative of the level of asthma medication treatment compliance by a user of the inhaler by indicating that the user is within a time window to take a dose.

11. The method of claim 7 wherein the signal is indicative of the level of asthma medication treatment compliance by either indicating in one way that the user of the inhaler missed a dose or indicating in a different way that the user of the inhaler missed multiple doses.

12. The method of claim 7 including outputting a second signal via an indicator joined with the dock if the inhaler to which the inhaler module was or is connected is low on medication or the inhaler is past an expiration date.

13. The method of claim 12 including outputting a third signal via an indicator joined with the dock when the inhaler module is downloading compliance data to the docking station.

14. The method of claim 13 including outputting a fourth signal via an indicator joined with the dock indicating the changing status of a battery in the inhaler module.

15. An apparatus for indicating mixing status of contents of a medication canister for an inhaler, the apparatus comprising:
an inhaler module that collects information related to use of the inhaler,
an accelerometer that generates accelerometer output signals in response to shaking;
a mounting element that removably mounts the inhaler module in a spatial location on the inhaler, wherein, when mounted, shaking the apparatus simultaneously shakes the contents of the medication canister and shakes the accelerometer;
an indicator for communicating information to a user of the apparatus;
a microprocessor in communication with the accelerometer, the microprocessor programmed to 1) determine a mixing status of the contents of the medication canister based on the accelerometer output signals and 2) control the indicator to indicate the mixing status to the user of the apparatus.

16. The apparatus of claim 15 wherein shaking the accelerometer wakes the inhaler module out of a sleep state.

17. The apparatus of claim 15 wherein the inhaler module is configured as a function of inhaler medication in the medication canister.

18. The apparatus of claim 15 wherein the microprocessor is programmed to control the indicator to prompt the user to puff the inhaler.

19. The apparatus of claim 15 wherein the microprocessor is programmed to initiate a countdown timer and to control the indicator to indicate the countdown timer to the user.

20. The apparatus of claim 15 wherein the microprocessor is programmed to determine whether the inhaler needs to be primed and control the indicator to indicate that the inhaler needs to be primed in response to the determination that the inhaler needs to be primed.

21. The apparatus of claim 15 wherein the microprocessor is programmed to determine whether a shake event occurs by comparing the accelerometer output to an acceleration threshold for a predetermined amount of time.

22. A method for operating an apparatus for monitoring compliance with a medication treatment regimen, the apparatus being attached to an inhaler including an medication canister including inhaler medication, the method comprising:
pairing an inhaler module with the inhaler, including configuring the inhaler module as a function of the inhaler medication;
shaking the inhaler to mix the inhaler medication in the medication canister before each use;
recording a timestamp in response to the shaking before each use;
determining whether the inhaler needs to be primed based on the recorded timestamps and the inhaler medication; and
in response to determining that the inhaler needs to primed, prompting the user to prime the inhaler.

23. The method of claim 22 wherein determining whether the inhaler needs to be primed based on the recorded timestamps and the inhaler medication includes determining whether the inhaler needs to be primed based on the difference between the timestamp recorded most recently and an internal clock.
